(19) United States
(12) Patent Application Publication
(10) Pub. No.: US 2013/0116583 A1
(43) Pub. Date: May 9, 2013

(54) SYSTEMS AND METHODS FOR PREDICTING AND CORROBORATING PULMONARY FLUID OVERLOADS USING AN IMPLANTABLE MEDICAL DEVICE

(71) Applicant: Xiaoyi Min, Thousand Oaks, CA (US)

(72) Inventor: Xiaoyi Min, Thousand Oaks, CA (US)

(73) Assignee: PACESETTER, INC., Sylmar, CA (US)

(21) Appl. No.: 13/288,704

(22) Filed: Nov. 3, 2011

(51) Int. Cl. A61B 5/0402; A61B 5/053

(52) U.S. Cl. 600/509; 600/547

(57) ABSTRACT

Techniques are provided for corroborating a preliminary detection of pulmonary fluid overload within a patient made initially based on transthoracic impedance. In one example, corroborative parameters pertaining to hematocrit, device pocket fluid accumulations, heart rate variability (HRV) and mean atrial tachycardia/atrial fibrillation (AT/AF) times are evaluated to confirm the fluid overload. Techniques are also provided for generating proxies for evaluating hematocrit and device pocket fluid accumulation based on certain impedance measurements. Still further, techniques are provided for predicting a possible pulmonary fluid overload based on trends in HRV or mean AT/AF times. System and method examples are set forth herein.

### FIELD OF THE INVENTION

The invention generally relates to implantable medical devices, such as pacemakers, implantable cardioverter/defibrillators (ICDs) and cardiac resynchronization therapy (CRT) devices and in particular to techniques for use by such devices within heart failure patients to predict and corroborate pulmonary fluid overloads due to pulmonary edema or other factors.

### BACKGROUND OF THE INVENTION

Heart failure is a debilitating disease in which abnormal function of the heart leads in the direction of inadequate blood flow to fulfill the needs of the tissues and organs of the body. Typically, the heart loses propulsive power because the cardiac muscle loses capacity to stretch and contract. Often, the ventricles do not adequately eject or fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds muscle causing the ventricles (particularly the left ventricle) to grow in volume in an attempt to pump more blood with each heartbeat. This places a still higher demand on the heart's oxygen supply. If the oxygen supply falls short of the growing demand, as it often does, further injury to the heart may result. The additional muscle mass may also stiffen the heart walls to hamper rather than assist in providing cardiac output. A particularly severe form of heart failure is congestive heart failure (CHF) wherein the weak pumping of the heart leads to build-up of fluids in the lungs and other organs and tissues.

Pulmonary edema is a swelling and/or fluid accumulation in the lungs often caused by heart failure. Briefly, the poor cardiac function resulting from heart failure can cause blood to back up in the lungs, thereby increasing blood pressure in the lungs, particularly pulmonary venous pressure. The increased pressure pushes fluid—but not blood cells—out of the blood vessels and into lung tissue and air sacs (i.e. the alveoli). This can cause severe respiratory problems and, left untreated, can be fatal. Pulmonary edema can also arise due to other factors besides heart failure, such as infections. Pulmonary edema due to heart failure may be referred to as cardiogenic pulmonary edema.

One technique for use by an implantable medical device for detecting pulmonary edema uses thoracic electrical impedance measurements to detect a “fluid overload,” i.e. a significant increase in thoracic fluids. A significant drop in thoracic impedance is deemed to be indicative of such a fluid overload. (In this regard, it is well known that worsening heart failure increases fluid retention and left atrial filling pressure so that the thoracic impedance is lowered.) In response, diuretics such as furosemide or bumetanide can be administered to the patient to reduce the fluid overload. Impedance-based techniques for detecting pulmonary edema are discussed in U.S. Pat. No. 7,272,443 to Min et al., entitled “System and Method for Predicting a Heart Condition based on Impedance Values using an Implantable Medical Device.” See, also, U.S. Pat. No. 8,032,212 of Bornzin et al., entitled “System and Method for Monitoring Thoracic Fluid Levels Based on Impedance Using an Implantable Medical Device” and U.S. Pat. No. 7,628,757 to Koh, entitled “System and Method for Impedance-Based Detection of Pulmonary Edema and Reduced Respiration using an Implantable Medical System.”

The use of thoracic impedance is promising since impedance can be readily measured in situ using implantable medical devices such as pacemakers or CRTs. However, impedance drops can occur within some patients without any clinical consequences and are often merely “false positive” fluid overload events. When titrating a patient with diuretics based on such drops in thoracic impedance, it is thus possible to overcorrect the fluid overload by dispensing too much diuretic. The patient may then become hypovolemic (a condition wherein there is too little blood). False positives may arise because there are multiple factors influencing thoracic impedance measurements including fluid factors (due pulmonary edema, pleural effusion, pericardial effusion and pneumonia), blood factors (due transfusions, changes in hematocrit, etc.) and factors due to surgical procedures performed on the patient.

Accordingly, it would be desirable to provide techniques for corroborating or confirming an impedance-based detection of a pulmonary fluid overload so as to reduce the risk of false positive detections. It is to this end that some aspects of the invention are directed.

False positives are particularly problematic in systems that do not adjust detection thresholds based on patient-specific information since impedance drops associated with fluid overloads can differ from patient to patient. See U.S. Pat. No. 7,774,055 to Min, entitled “Left atrial Pressure-based Criteria for Monitoring Intrathoracic Impedance,” and U.S. Pat. No. 7,574,255 also to Min, entitled “Criteria for Monitoring Intrathoracic Impedance,” which both discuss techniques for individualizing an impedance detection threshold based on patient heart failure status (using such parameters as a left atrial pressure (LAP), New York Heart Association (NYHA) classification, echocardiographic information, etc.) It would be desirable to provide further techniques for individualizing impedance-based detection of a pulmonary fluid overload using patient-specific information and it is to this end that other aspects of the invention are directed.

Still other aspects of the invention are directed to detecting hematocrit or pocket fluid accumulation, both of which are relevant to reliable fluid overload detection. The hematocrit (Ht or HCT)—also referred to as packed cell volume (PCV) or erythrocyte volume fraction (EVF)—is the percentage of blood volume occupied by red blood cells. Pocket fluid accumulation refers to the accumulation of fluids around the housing of the implantable device within its subcutaneous tissue pocket. Both hematocrit and device pocket fluids can affect thoracic impedance measurements. Still further aspects of the invention are directed to predicting a pulmonary fluid overload before it occurs so that suitable warnings or other actions can be taken.

### SUMMARY OF THE INVENTION

In accordance with an exemplary embodiment of the invention, techniques are provided for use with an implantable medical device for implant within a patient for corroborating a pulmonary fluid overload detection. In one example, transthoracic impedance is detected within the patient, then the device detects a possible pulmonary fluid overload based on a drop in transthoracic impedance. For example, the device calculates a fluid index based on transthoracic impedance and compares the index to a threshold to detect a “fluid index crossing” indicative of a possible fluid overload. In response, the device detects corroborative parameters such as hematocrit proxies or device pocket fluid accumulation proxies, then confirms or disconfirms the indication of pulmonary edema using the corroborative parameters. For example, if the proxy for hematocrit is found to have recently changed, then the drop in transthoracic impedance might be due to the change in hematocrit rather than an actual fluid overload and so the detection of the possible overload made based on the drop in transthoracic impedance is disconfirmed. That is, the initial impedance-based pulmonary fluid overload detection may have been a false positive. Conversely, a detection of no significant change in hematocrit would tend to confirm the fluid overload. As another example, if device pocket fluids have recently increased, then the drop in transthoracic impedance might be due to the pocket fluids and so the initial detection of fluid overload is likewise deemed to be a false positive. On the other hand, the detection of no significant change in pocket fluids would tend to confirm the fluid overload. If the initial detection of fluid overload is confirmed, the device generates an indication of the overload and, if equipped with a drug pump, the implantable system automatically delivers diuretics or other suitable compounds to mitigate the overload. In other implementations, the system transmits information to an external device (such as a bedside monitor or hand-held interface device) for notifying the patient or caregiver of the need to adjust the dosage of diuretics or other medications. In this manner, false positives are reduced or avoided by corroborating the fluid overload detection based on corroborative parameters such as hematocrit or pocket fluid proxies. Other parameters that may be used to corroborate a fluid overload include heart rate variability (HRV) and the duration of atrial tachycardia/atrial fibrillation (AT/AF) episodes.

In an illustrative embodiment, where the device is a pacemaker, ICD or CRT equipped with various pacing/sensing leads, the device exploits intracardiac impedance (IACZ) signals measured using an RV lead to generate a proxy or surrogate for hematocrit for use in corroborating the initial detection of a pulmonary fluid overload. In one example, the device inputs patient-specific calibration information relating baseline hematocrit to baseline IACZ within the patient measured along either an RV tip—RV ring vector or an RV coil—RV ring vector (herein “RV IACZ”.) RV IACZ tends to emphasize local impedance changes due to changes in the blood and hence is influenced by hematocrit. The calibration information may be generated under clinician supervision about six weeks following device implant by withdrawing blood from the patient and measuring hematocrit using otherwise conventional techniques. The measured value of hematocrit is stored in a memory system of the device along with corresponding RV IACZ values detected within the patient at the same time. The calibration information thereby relates baseline hematocrit to baseline RV IACZ for the patient. The RV IACZ values are preferably measured at the peak of the QRS complex of an intracardiac electrogram (IEGM.) Thereafter, during routine device operation, the device measures and tracks RV IACZ within the patient using the same vector (also preferably at the peak of the QRS) and then generates a proxy for hematocrit by comparing the measured RV IACZ against the patient-specific calibration information. In general, if hematocrit increases, then blood resistivity increases and so RV IACZ also typically increases, other factors being unchanged. Conversely, if hematocrit decreases, RV IACZ typically decreases, other factors being unchanged. Accordingly, RV IACZ may be used as proxy for hematocrit so that changes in hematocrit can be detected based on changes in the RV IACZ. Cardiac dimension change is an important factor for RV IACZ and so measurements are preferably made at the end of diastolic phase of a cardiac cycle i.e. at peak of QRS so that RV is in full of blood around RV IACZ. (Note that different RV IACZ calibration values may obtained at different patient postures or at different activity levels to thereby improve the specificity by which RV IACZ serves as a proxy for hematocrit. Alternatively, RV IACZ values at peak of QRS may be collected periodically over a day and then averaged so as to average out variations due to posture or activity or other such factors.) In one example, whenever the device detects a possible pulmonary fluid overload based on transthoracic impedance (as measured RV coil to device housing (i.e. “can”)), the device then examines recent changes, if any, in RV IACZ. If RV IACZ has dropped significantly, the detection of the fluid overload is deemed to be a false positive and no diuretics are delivered. Rather, a warning of anemia may be generated. If RV IACZ has not dropped significantly, then the detection of the fluid overload is deemed to be reliable (at least insofar as hematocrit corroboration is concerned) and diuretics are delivered and appropriate warnings are generated to alert the patient or caregiver.

In another illustrative embodiment, the device exploits transthoracic impedance signals measured between a superior vena cava (SVC) coil electrode and the device housing (i.e. can) to estimate and track pocket fluids for use in corroborating a pulmonary fluid overload detection. As with the hematocrit example above, the device inputs patient-specific calibration information. In this example, the calibration information includes baseline SVC coil—can impedance measurements taken after initial device implant during a period of time when device pocket fluid accumulation is expected due to healing from the surgery used to implant the device. (In the case of device replacement, baseline SVC coil—can impedance measurements are preferably made before the surgery to replace the device and may be regarded as “pre-surgery” measurements.) The baseline SVC coil—housing impedance values (herein “SVC coil—can Z”) are stored in the device. (As with the RV IACZ values discussed above, the SVC coil—can Z values are also preferably measured at the peak of the QRS complex of the IEGM.) Thereafter, during routine device operation, the device measures and tracks SVC—can Z within the patient (also preferably at the peak of the QRS) for use as a proxy or surrogate for pocket fluid accumulation. In general, during the weeks or months following implant, the initial post-surgery fluid accumulation in the device pocket should dissipate so that SVC coil—can Z should therefore increase. Any subsequent decrease in SVC coil—can Z indicates that fluid may be accumulating once again in the device pocket for noncardiogenic reasons, perhaps as the result of an infection. Accordingly, in one example, whenever the device detects a possible pulmonary fluid overload based on transthoracic impedance (measured RV coil—can), the device then examines recent changes, if any, in SVC coil—can Z. If SVC coil—can Z has recently decreased significantly, then the detection of the fluid overload is deemed to be a false positive and no diuretics are delivered. Rather, a warning of pocket fluid accumulation may be generated. If SVC—can Z does not exhibit any recent and significant changes, then the detection of the fluid overload is deemed to be reliable (at least insofar as pocket fluids are concerned) and diuretics are delivered and appropriate warnings are generated to alert the patient or caregiver. As with the RV IACZ example above, different calibration values may obtained for SVC coil—can Z at different patient postures or at different activity levels to thereby improve the specificity by which SVC coil—can Z serves as a proxy for pocket fluid accumulation.

In yet another illustrative embodiment, the device exploits mean AT/AF times within patient to corroborate the pulmonary fluid overload detection wherein the mean AT/AF time is representative of the total duration of spontaneous AT/AF episodes in the patient divided by the corresponding follow-up time. This may also be regarded as an “AT/AF burden.” That is, during device operation, the device detects and tracks episodes of AT and AF and assesses the accumulated duration or time of individual episodes relative to corresponding follow-up times so as to assess the mean AT/AF time. The mean AT/AF can be represented by days from the start of the follow-up. The device also tracks changes, if any, in the mean AT/AF times to determine a rate of change in mean AT/AF times (i.e. the device determines the slope of a mean AT/AF time curve.) It has been found that episodes of cardiogenic pulmonary fluid overloads (as detected by a fluid index crossing) are preceded by increases in mean AT/AF times and so the slope of the AT/AF curve is typically fairly large prior to fluid index crossings. Accordingly, if the slope of the AT/AF curve is low or negative, the fluid overload indicated by the fluid index crossing is likely a false positive. If the slope of the AT/AF curve is relatively high, the fluid overload is corroborated. Hence, in one example, whenever the device detects a possible pulmonary fluid overload, the device determines the slope of the mean AT/AF time curve during the weeks prior to the event and compares the slope against an AT/AF slope threshold. If the AT/AF slope exceeds the threshold, the fluid overload is confirmed.

In still another illustrative embodiment, the device exploits HRV to corroborate the pulmonary fluid overload detection. During device operation, the device detects and tracks HRV using otherwise conventional techniques. The device also tracks changes, if any, in HRV to determine a rate of change in HRV. It has been found that episodes of cardiogenic pulmonary edema (as detected by fluid index crossings) are preceded by decreases in HRV and so the slope of an HRV curve is typically strongly negative prior to fluid index crossings. Accordingly, if the slope of the HRV curve is flat or positive, the fluid overload indicated by the fluid index crossing is likely a false positive. If the slope of the HRV curve is strongly negative, the fluid overload is corroborated. Hence, in one example, whenever the device detects a possible pulmonary fluid overload, the device then determines the slope of the HRV curve and compares the slope against a negative HRV slope threshold. If the HRV slope is below the threshold (i.e. the downward HRV slope is steeper than the negative slope threshold), the fluid overload is confirmed.

In some examples, all four of the corroborative parameters (hematocrit proxies, pocket fluid proxies, mean AT/AF times and HRV) are employed to corroborate the detection of a fluid overload before therapy is delivered and warnings are generated. In other examples, only one or two of the corroborative parameters are exploited. In any case, by using these parameters to corroborate detection of fluid overload, false positives are reduced or eliminated.

In still other examples, the device exploits the aforementioned techniques to detect changes in hematocrit or device pocket fluids for purposes other than fluid overload corroboration. Briefly, in one example, the device assesses hematocrit by: inputting patient-specific calibration information relating baseline hematocrit to baseline RV IACZ within the patient; measuring RV IACZ within the patient; and then generating a proxy for hematocrit by comparing the measured RV IACZ against the patient-specific calibration information relating baseline hematocrit to baseline RV IACZ for the patient. In response to any significant change in the hematocrit proxy, the device can control various device functions such as issuing warnings of possible anemia. In another example, the device assesses changes in pocket fluids by: inputting patient-specific calibration information pertaining to baseline SVC coil—can Z transthoracic impedance within the patient; measuring SVC coil—can Z transthoracic impedance within the patient; and then generating a proxy for pocket fluid accumulation by comparing the measured impedance against the patient-specific calibration information. In response to any significant change in the pocket fluid proxy, the device can control various device functions such as issuing warnings of possible fluid accumulations.

Still further, the device can be equipped to predict a pulmonary fluid overload based on mean AT/AF times or HRV. In one example, the device predicts a possible fluid overload based on mean AT/AF times by: detecting spontaneous episodes of AT/AF and measuring the durations of individual episodes and corresponding follow-up times; determining mean AT/AF times based on a total duration of spontaneous AT/AF episodes in the patient divided by the corresponding follow-up times; detecting a rate of change, if any, in the mean AT/AF times; and then predicting a possible fluid overload within the patient based the rate of change in the mean AT/AF times. As noted above, the slope of the mean AT/AF time curve typically increases prior to an episode of cardiogenic pulmonary edema. Hence, a significant increase in the slope (i.e. the rate of change) of the AT/AF curve is predictive of a possible fluid overload. In another example, the device predicts a possible pulmonary fluid overload based on HRV by: detecting HRV within the patient; detecting a rate of change, if any, in HRV; and then predicting a fluid overload within the patient based the rate of change in HRV. As also explained, the slope of the HRV curve can decrease sharply prior to an episode of cardiogenic pulmonary edema. Hence, a significant drop in the slope (i.e. the rate of change) of the HRV curve is predictive of a possible fluid overload. In response to the prediction of a possible fluid overload, warnings may be generated to alert the patient or caregiver or other steps may be taken.

System and method examples of various embodiments of the invention are described herein.

### BRIEF DESCRIPTION OF THE DRAWINGS

The above and further features, advantages and benefits of the invention will be apparent upon consideration of the present description taken in conjunction with the accompanying drawings, in which:

FIG. 1 illustrates pertinent components of an implantable medical system having a pacemaker or ICD capable of predicting pulmonary fluid overloads and/or corroborating the detection of a fluid overload within a patient;

FIG. 2 summarizes a pulmonary fluid overload corroboration technique for confirming the detection of a fluid overload made based on transthoracic impedance, which may be performed by the system of FIG. 1;

FIG. 3 illustrates an exemplary embodiment of the corroboration technique of FIG. 2 wherein corroboration is based on a hematocrit proxy;

FIG. 5 illustrates an exemplary embodiment of the corroboration technique of FIG. 2 wherein corroboration is based on a pocket fluid proxy;

FIG. 5 illustrates an exemplary embodiment of the corroboration technique of FIG. 2 wherein corroboration is based on mean AT/AF times;

FIG. 6 is a graph illustrating an AT/AF curve relating mean AT/AF times relative to a fluid index crossing time exploited by the technique of FIG. 5;

FIG. 7 illustrates an exemplary embodiment of the corroboration technique of FIG. 2 wherein corroboration is based on HRV;

FIG. 8 is a graph illustrating an HRV curve relating HRV to a fluid index crossing time exploited by the technique of FIG. 7;

FIG. 9 illustrates an exemplary combined embodiment of the corroboration technique of FIG. 2 wherein corroboration is based on hematocrit proxies, pocket fluid proxies, mean AT/AF times and HRV;

FIG. 10 summarizes a technique for generating a proxy for hematocrit based on RV IACZ, which may be performed by the system of FIG. 1;

FIG. 11 summarizes a technique for generating a proxy for pocket fluid accumulation based on SVC coil—can impedance, which may be performed by the system of FIG. 1;

FIG. 12 summarizes a technique for predicting a fluid overload based on mean AT/AF times, which may be performed by the system of FIG. 1;

FIG. 13 summarizes a technique for predicting a fluid overload based on HRV, which may be performed by the system of FIG. 1;

FIG. 14 is a simplified, partly cutaway view, illustrating the pacer/ICD of FIG. 1 along with a set of leads implanted on or in the heart of the patient; and

FIG. 15 is a functional block diagram of the pacer/ICD of FIG. 14, illustrating basic circuit elements that provide cardioversion, defibrillation and/or pacing stimulation in the heart and particularly illustrating components for performing or controlling the techniques of FIGS. 2-13.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System

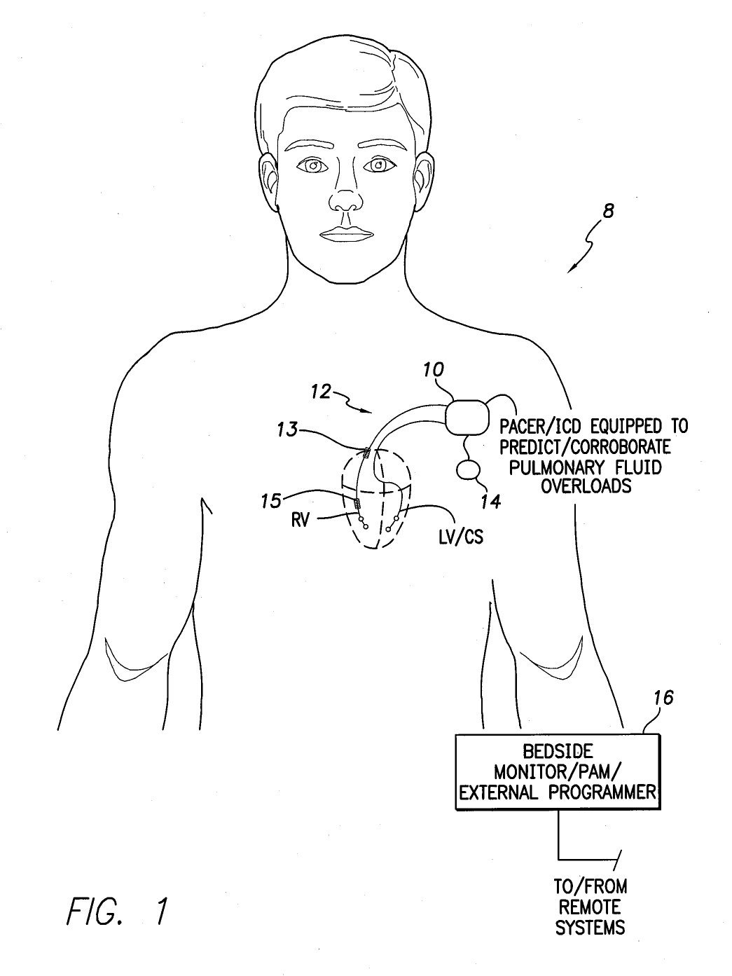

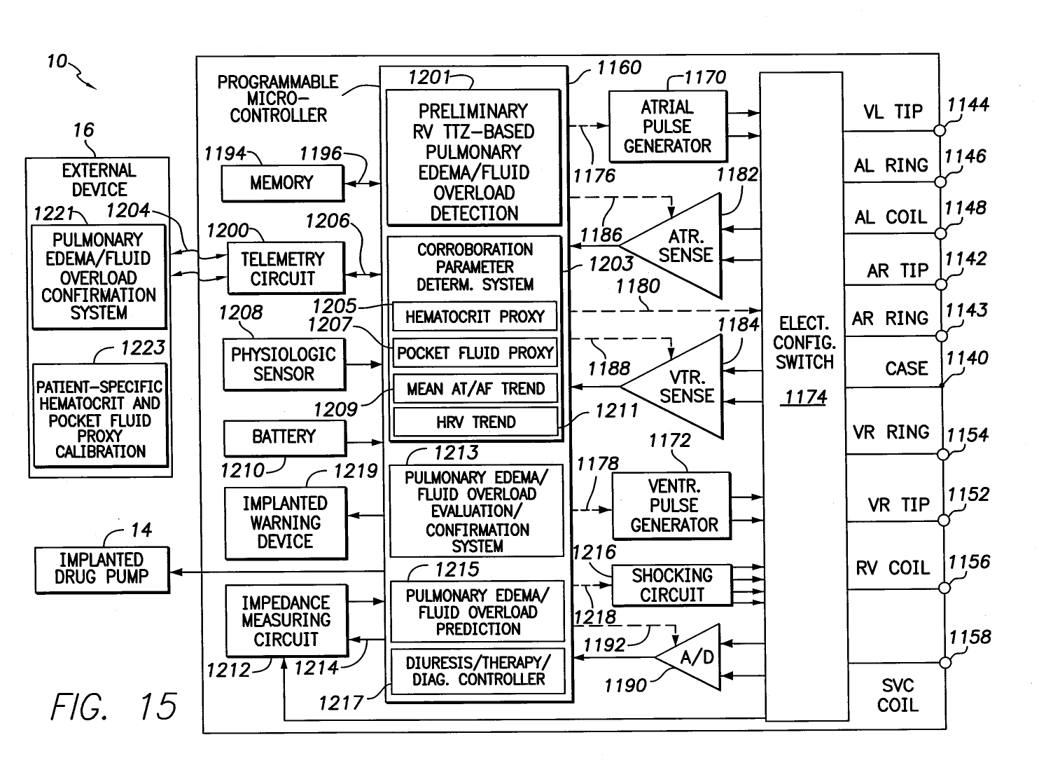

FIG. 1 illustrates an implantable medical system 8 capable of detecting predicting and corroborating a pulmonary edema fluid overload based on various parameters such as HRV or mean AT/AF times. The system is also capable of titrating dosages of diuretics or other medications in response thereto, as well as performing other therapeutic or diagnostic functions. Still further, the system may be equipped to detect changes in hematocrit or device pocket fluids. To these ends, medical system 8 includes a pacer/ICD/CRT 10 or other cardiac rhythm management device equipped with one or more cardiac sensing/pacing leads 12 implanted within the heart of the patient. In FIG. 1, two exemplary leads are shown: a bipolar RV lead and a bipolar LV lead implanted via the coronary sinus (CS). An RA lead may also provided that includes a bipolar RA tip/ring pair. Other suitable leads may instead be employed, including leads with more or fewer electrodes, such as quad-pole leads. Also, as shown, the exemplary RV lead has an SVC coil electrode 13 implanted in or near the SVC and a larger RV coil 15 implanted within the RV itself. Other electrodes of various sizes and shapes may be additionally or alternatively provided, such as an LV coil. Amore complete set of leads is illustrated in FIG. 15. Although identified as a “pacer/ICD/CRT” in FIG. 1, it should be understood that device 10 can be any suitably-equipped implantable medical device, such as a standalone pacemaker, ICD or CRT device, including CRT-D and CRT-P devices. In the following, for brevity, device 10 will be referred to simply as a pacer/CRT.

In examples where the system is intended to automatically titrate diuretics based on pulmonary fluid accumulations, an implanted or subcutaneous drug pump or other drug dispensing device 14 may be used, which is controlled by the pacer/CRT. Implantable drug pumps for use in dispensing medications are discussed in U.S. Pat. No. 5,328,460 to Lord et al., entitled “Implantable Medication Infusion Pump Including Self-Contained Acoustic Fault Detection Apparatus.”

In other embodiments, information pertaining to any pulmonary fluid overloads is transmitted to an external system 16, which generates diagnostic displays instructing the patient (directly or via a caregiver) to take certain dosages of diuretics or other medications. System 16 may include, for example, an external programmer, bedside monitor or hand-held personal advisory module (PAM). Data from the external system can be forwarded to a centralized system such as the Merlin.Net system, the HouseCall™ remote monitoring system or the Merlin@home systems of St. Jude Medical for notifying the clinician of a pulmonary fluid overload within the patient.

Warnings as to pulmonary fluid overloads may also be generated using the bedside monitor, PAM, or an internal warning device provided within the pacer/ICD. The internal warning device (which may be part of pacer/CRT) can be a vibrating device or a “tickle” voltage device that, in either case, provides perceptible stimulation to the patient to alert the patient. (Again, see Lord et al.) The bedside monitor or PAM can provide audible or visual alarm signals to alert the patient or caregiver, as well as appropriate textual or graphic displays. In addition, diagnostic information pertaining to changes in pulmonary fluid levels (and to any medical conditions detected therefrom) may be stored within the pacer/CRT for subsequent transmission to an external programmer (not shown in FIG. 1) for review by a clinician during a follow-up session between patient and clinician. The clinician then prescribes any other appropriate therapies to address the condition. The clinician may also adjust the operation of the pacer/CRT to activate, deactivate or otherwise control any therapies provided.

Additionally, the pacer/CRT can perform a wide variety of pacing and/or defibrillation functions such as delivering pacing in response to an arrhythmia or generating and delivering shocks in response to fibrillation. As a pacer/CRT, the device is also equipped to deliver and control CRT. Briefly, CRT seeks to normalize asynchronous cardiac electrical activation and resultant asynchronous contractions associated with CHF by delivering synchronized pacing stimulus to both ventricles. The stimulus is synchronized so as to improve overall cardiac function. This may have the additional beneficial effect of reducing the susceptibility to life-threatening tachyarrhythmias. CRT is discussed, for example, in U.S. Patent Application 2008/0306567 of Park et al., entitled “System and Method for Improving CRT Response and Identifying Potential Non-Responders to CRT Therapy.” See, also, U.S. Pat. No. 7,912,544 to Min et al., entitled “CRT Responder Model using EGM Information” and U.S. Pat. No. 7,881,787 to Min, entitled “Capture Detection System and Method CRT Therapy.”

Note that systems provided in accordance with the invention need not include all the components shown in FIG. 1. In many cases, for example, the implantable system will include only the pacer/CRT and its leads. Drug pumps are not necessarily employed. Some implementations may employ an external monitor for generating warning signals but no internal warning device. These are just a few exemplary embodiments. No attempt is made herein to describe all possible combinations of components that may be provided in accordance with the general principles of the invention. Also, note that, although internal signal transmission lines are shown in FIG. 1 for interconnecting implanted components, wireless signal transmission might alternatively be employed. In addition, it should be understood that the particular shape, size and locations of the implanted components shown in FIG. 1 are merely illustrative and may not necessarily correspond to actual implant locations. In particular, preferred implant locations for the leads are more precisely illustrated in FIG. 15.

In the following, techniques for corroborating a pulmonary fluid overload detection will be described in detail first. Then, techniques for assessing hematocrit or fluid pocket accumulation proxies will described, followed by techniques for predicting possible pulmonary fluid overloads based on HRV or mean AT/AF times.

### Pulmonary Edema/Fluid Overload Corroboration Techniques

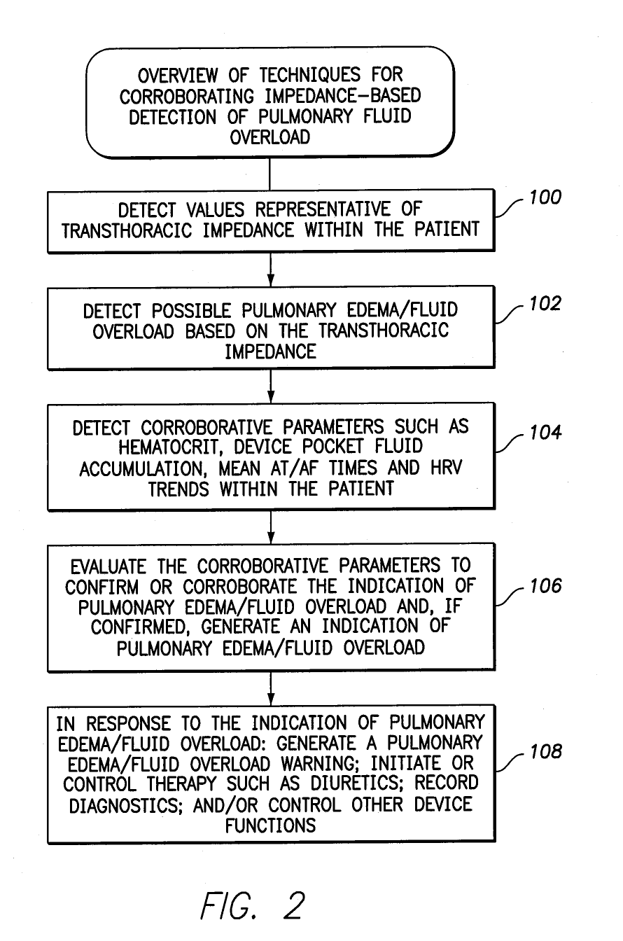

FIG. 2 broadly summarizes general techniques for corroborating a detection of pulmonary fluid overload that may be exploited by the pacer/ICD of FIG. 1 or other suitably equipped systems. Beginning at step 100, the pacer/ICD detects values representative of transthoracic impedance (TTZ) within the patient along a vector selected to correlate TTZ with pulmonary fluid overloads, such as a vector from the RV coil to the device housing (i.e. the can.) Insofar as impedance is concerned, it should be understood that related electrical parameters might be detected and/or exploited instead, such as admittance, conductance or immittance. Those skilled in the art can convert among these related parameters where appropriate. Herein, “values representative of impedance” is intended to generally include related electrical parameters. At step 102, the device detects a possible pulmonary edema/fluid overload event based on the transthoracic impedance. This may be achieved, for example, by measuring transthoracic impedance values, averaging the values and then detecting a sharp drop in average transthoracic relative to a baseline transthoracic by comparison with a suitable threshold. Impedance-based pulmonary edema/fluid overload detection techniques are discussed in the patent documents of Min et al., Bornzin et al. and Koh cited above. See, also, U.S. Pat. No. 8,010,196 to Wong et al., entitled “Tissue Characterization using Intracardiac Impedances with an Implantable Lead System,” which describes a frequency-rich, low energy, multi-phasic waveform for use in measuring impedance.

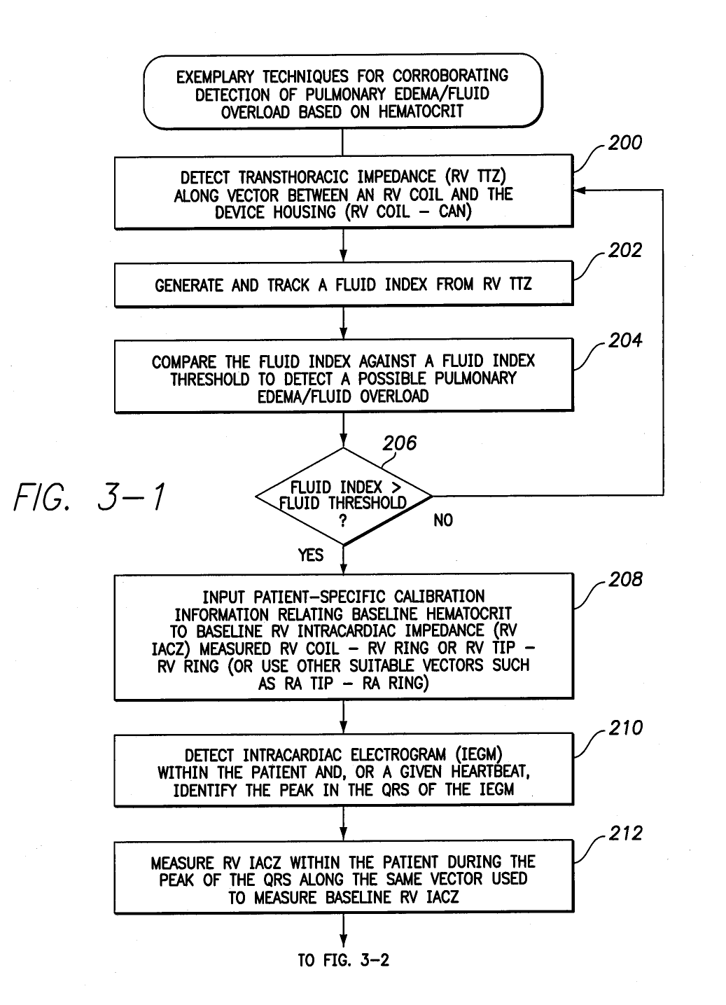

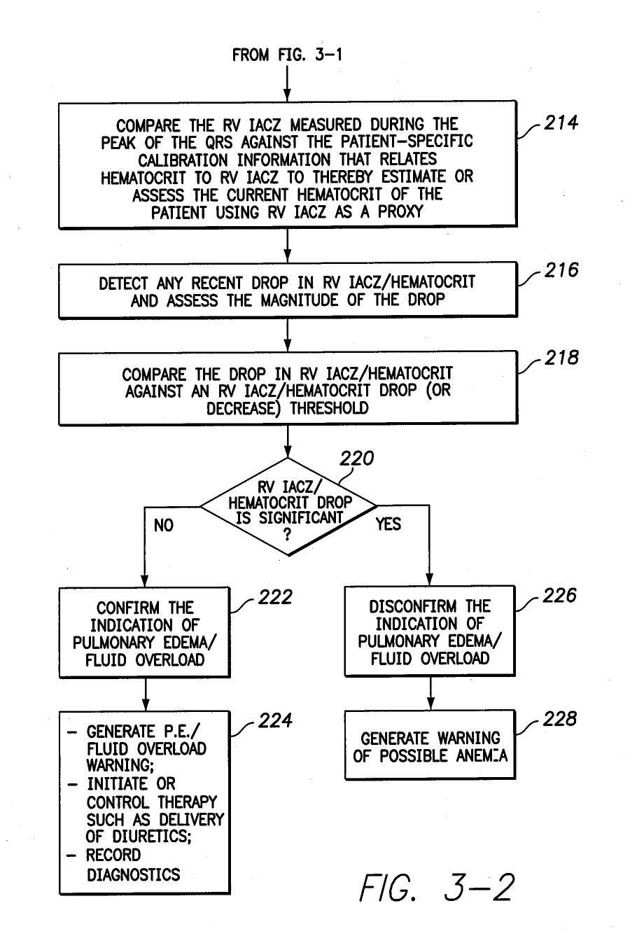

At step 104, the device then detects one or more corroborative parameters such as hematocrit (directly or via a proxy), device pocket fluid accumulation (directly or via a proxy), mean AT/AF times and HRV trends within the patient. At step 106, the device then evaluates the corroborative parameters to confirm or corroborate the indication of pulmonary edema/fluid overload and, if confirmed/corroborated, the device generates an indication of pulmonary edema/fluid overload. The indication can be, for example, a warning indicator issued to the patient or caregiver or it can be an internal notification signal or data value for use within the device to trigger various device functions. Accordingly, at step 108, in response to the indication of pulmonary edema/fluid overload, the device: generates a pulmonary edema/fluid overload warning; initiates or controls therapy such as diuretics; records diagnostics; and/or performs or controls other suitable device functions, alone or in combination with other devices. The corroboration techniques broadly summarized in FIG. 2 will be described in greater detail within FIGS. 3-9.

FIG. 3 illustrates corroboration techniques that exploit hematocrit proxies. At step 200, the device detects transthoracic impedance along the RV coil—can vector (herein RV TTZ) and, at step 202, generates and tracks a fluid index, which is derived from RV TTZ based on averaged values for RV TTZ. RV TTZ may be averaged, for example, over a period of a day to substantially average out variations due to circadian rhythms, patient posture, patient activity, etc. That is, an assembled running average or daily average can be used. In one example, the fluid index is calculated based on an accumulation of consecutive day-to-day differences between a daily RV TTZ value and a reference RV TTZ value. The fluid index may be reset when the lungs are drying out. In the examples described herein the fluid index is defined as a value that increases with increasing fluid accumulation and decreases with decreasing fluid amounts. Hence, a larger fluid index means more fluids and vice versa. (Note that the actual impedance measurements obtained by the device will tend to decrease with increasing fluid accumulations and vice versa, since the presence of fluids between the measuring electrodes will tend to lower the electrical impedance.)

For a discussion of a fluid index, see, for example, U.S. Patent Application 2009/0270746 of Min, entitled “Criteria for Monitoring Intrathoracic Impedance” and U.S. Pat. No. 7,574,255, of the same title. See, also, U.S. Pat. No. 7,774,055 to Min, entitled “Left Atrial Pressure-Based Criteria for Monitoring Intrathoracic Impedance,” and U.S. Patent Application 2010/0305641 of Pillai et al., entitled “System and Method for Detecting Pulmonary Edema based on Impedance Measured using an Implantable Medical Device during a Lead Maturation Interval.”

At step 204, the device compares the fluid index against a fluid index threshold to detect a possible pulmonary edema/fluid overload. In this regard, a sharp increase in the fluid index is deemed indicative of a possible pulmonary edema/fluid overload event within the patient. The threshold may be set relative to a baseline fluid index for the patient, subject to the discretion of the clinician. So long as the fluid index remains at or below the threshold (as determined at decision step 206), the device repeats steps 200-204 to periodically update and assess the fluid index. This may be performed, for example, once a day.

Assuming, though, that the fluid index rises above the threshold, then at step 208 the device inputs patient-specific calibration information from its memory systems that relate baseline hematocrit to baseline RV IACZ within the patient wherein the baseline RV IACZ is measured RV coil—RV ring or RV tip—RV ring. Alternatively, other suitable vectors may be used such as RA tip—RA ring. It is noted that when using an RA tip—RA ring vector, the resulting IACZ value is an RA IACZ value rather than an RV IACZ value and so, in the following descriptions, it should be understood that RA IACZ could be used in place of RV IACZ, where appropriate. As summarized above, the calibration information may be initially generated by a clinician several weeks following device implant (e.g. six weeks) by withdrawing blood from the patient and measuring hematocrit using otherwise conventional techniques. The measured value of the hematocrit is stored in a memory system of the device along with baseline RV IACZ values detected within the patient at about the same time. The calibration information thereby relates baseline hematocrit to baseline RV IACZ for the patient. Note also that baseline RV IACZ values are preferably measured at the peak of the QRS complex of the IEGM.

After inputting the patient-specific calibration information, the device at step 210 then detects the current IEGM for the patient and, for a given heartbeat, the device identifies the peak in the QRS of the IEGM (i.e. the peak of the ventricular depolarization waveform.) At step 212, the device measures RV IACZ within the patient during the peak of the QRS along the same vector used to measure baseline RV IACZ. (Note that the RV IACZ values are preferably measured over several heartbeats (e.g. eight beats) over several respiration cycles in accordance with a predetermined daily schedule, then averaged over each day. If so, then the daily averaged RV IACZ values are used in the following as the RV IACZ values.) At step 214, the device compares the RV IACZ measured during the peak of the QRS against the patient-specific calibration information that relates baseline hematocrit to baseline RV IACZ to thereby assess or estimate the current hematocrit of the patient using RV IACZ as a proxy. In general, if hematocrit increases, RV IACZ measured also increases, other factors being unchanged. Conversely, if hematocrit decreases, RV IACZ decreases, other factors being unchanged. Accordingly, RV IACZ may be used as proxy for hematocrit so that significant changes in hematocrit can be detected based on changes in the RV IACZ. (Note that different RV IACZ calibration values may obtained at different patient postures or at different activity levels to thereby improve the specificity by which RV IACZ serves as a proxy for hematocrit.) Alternatively, baseline tests can be made at different postures or activity levels to establish the sensitivity of RV IACZ. If the variation in RV IACZ is 10%, for example, the threshold would be set or adjusted to take this variation into account. Trimming of the daily average for baseline can be exploited for comparisons to newly measured daily average values for RV IACZ. In some embodiments, the device may be programmed to actually estimate hematocrit from RV IACZ to yield a percentage value representative of the estimated percentage of patient blood occupied by red blood cells. Thereafter, the estimated hematocrit value is compared against a hematocrit threshold. This, however, is not necessary as it is sufficient for the purposes of the technique of FIG. 3 to use RV IACZ as a proxy for hematocrit. That is, a drop in the value of RV IACZ (represented as a percentage drop) can be compared against an RV IACZ threshold (represented as a percentage drop threshold) without the need to actually estimate hematocrit. Herein, the term “RV IACZ/hematocrit” is used to generally indicate that either RV IACZ values or hematocrit values may be used, depending upon the programming of the device. Note that value for the RV IACZ/hematocrit drop threshold can be initially set, for example, to a default value set such as −10%. The threshold can thereafter be adjusted based on RV IACZ/hematocrit collected within the patient prior to known and verified pulmonary edema/fluid overloads.

At step 216, the device detects any recent drop in RV IACZ/hematocrit and assesses the magnitude of the drop and then, at step 218, compares the drop in RV IACZ/hematocrit against an RV IACZ/hematocrit drop threshold. That is, the threshold assesses the amount or magnitude of any drop in RV IACZ/hematocrit. The threshold is exceeded if the drop is relatively large. The threshold is not exceeded if the drop is relatively small or if there is no drop. If RV IACZ/hematocrit has dropped significantly, then the detection of the fluid overload is deemed to be a false positive since the fluid overload detected based on RV TTZ may have been due to the drop hematocrit (e.g. due to anemia) rather than any increase in pulmonary fluids. (In some examples, to disconfirm a fluid overload detection, the drop in RV IACZ/hematocrit must remain greater than the corresponding threshold for at least five days. This helps avoid declaring a false positive too quickly.) Conversely, if RV IACZ/hematocrit has not dropped significantly, then the detection of the fluid overload is deemed to be reliable (at least insofar as hematocrit is concerned.) Accordingly, following step 220, if no significant drop in RV IACZ/hematocrit has been detected, then the indication of a possible pulmonary fluid overload initially made at step 206 is deemed to be confirmed or corroborated at step 222 (or at least has not been disconfirmed.)

Thereafter, at step 224, the device responds by: generating a pulmonary edema/fluid overload warning; initiating or controlling therapy such as the delivery of diuretics; recording diagnostic; and/or controlling or triggering other suitable device functions. On the other hand, if a significant drop in RV IACZ/hematocrit was detected, then the indication of a possible pulmonary fluid overload made at step 206 is deemed to be disconfirmed at step 226 (or at least not corroborated.) At step 228, the device may generate a warning of possible anemia in view of the drop in RV IACZ/hematocrit. Note that, if a significant change in hematocrit is detected, a correction term can be applied to subsequent ΔZ measurements employed by the device, e.g. ΔZ=ΔZ_TTZ−αΔZ_hematocrit where α is a coefficient based on the change in TTZ from hematocrit.

Note also that, rather than employ RV IACZ as a proxy for hematocrit, the device can instead measure or estimate hematocrit using other techniques. Techniques for assessing hematocrit are discussed in: U.S. Pat. No. 7,920,913 to Nabutovsky et al., entitled “Systems and Methods for Increasing Implantable Sensor Accuracy”; U.S. Pat. No. 7,654,964 to Kroll et al., entitled “System and Method for Detecting Arterial Blood Pressure based on Aortic Electrical Resistance using an Implantable Medical Device”; and U.S. Patent Application 2011/0098546 of Farazi et al., entitled “Assessing Medical Conditions based on Venous Oxygen Saturation and Hematocrit Information.” See, also, U.S. patent application Ser. No. 11/927,026, of Nabutovsky et al., filed Oct. 29, 2007, entitled “Systems and Methods for Exploiting Venous Blood Oxygen Saturation in combination with Hematocrit or other Sensor Parameters for use with an Implantable Medical Device.”

One particular example of the hematocrit corroboration technique of FIG. 3 is summarized as follows:

Store Baseline IACZ with known hematocrit:
6 weeks post implant, check hematocrit at office visit
If a RV coil is available, use RV coil to RV ring; otherwise use RV tip to RV ring
Measure intra-cardiac Z of RV Coil to RV ring timed at peak of QRS for several beats (8 beats) over several respiration cycles
Store a daily average for the same day at predetermined times over the day.

Monitoring:
At the time of taking intra-thoracic Z (i.e. RV TTZ), measure intra-cardiac Z (i.e. RV IACZ)
Store daily average RV IACZ over time that can be plotted or stored as a surrogate of hematocrit and for predicting anemia.
If RV TTZ has fluid index threshold crossing, check the RV IACZ changes. If the drop in RV IACZ is greater than the threshold, a warning of edema would be false positive.

FIG. 4 illustrates corroboration techniques that exploit pocket fluids. Some aspects of the technique are the same or similar to those described with reference to FIG. 3 and hence will not be described again in detail. At step 300, the device detects RV TTZ along the RV coil—can vector and, at step 302, generates and tracks the fluid index. At step 304, the device compares the fluid index against the fluid index threshold to detect a possible pulmonary edema/fluid overload. As longs as the fluid index remains at or below the threshold (as determined at decision step 306), the device repeats steps 300-304 to periodically update and assess the fluid index. Assuming, though, that the fluid index rises above the threshold, then at step 308 the device inputs patient-specific calibration information from its memory systems that relate baseline transthoracic impedance measured SVC coil—can following implant. The calibration information may be initially generated following device implant by measuring and recording the SVC coil—can Z, preferably at the peak of the QRS complex of the IEGM. Note that different SVC coil—can Z calibration values may obtained at different patient postures or at different activity levels to thereby improve the specificity by which SVC coil—can Z serves as a proxy for pocket fluids.

After inputting the patient-specific calibration information, the device at step 310 then detects the peak of the QRS complex within heartbeats of the current IEGM. At step 312, the device measures SVC coil—can Z within the patient during the peak of the QRS for comparison against the baseline values input at step 308. At step 314, the device compares the newly detected SVC coil—can Z values against the baseline SVC coil—can Z values to assess differences indicative of pocket fluid accumulation (i.e. the device uses SVC coil—can Z as a proxy or surrogate for changes in pocket fluids.) In general, during the weeks or months following implant, initial fluid accumulation in the device pocket should dissipate so that SVC coil—can Z should increase. Any subsequent decrease in SVC coil—can Z indicates that fluid may be accumulating once again in the device pocket for noncardiogenic reasons, perhaps as the result of an infection. Accordingly, SVC coil—can Z may be used as proxy for pocket fluid accumulation.

At step 316, the device detects any recent drop in SVC coil—can Z and assesses the magnitude of the drop (such as a percentage drop) and then, at step 318, compares the drop against an SVC coil—can Z drop threshold. That is, the threshold assesses the amount or magnitude of any drop in SVC coil—can Z. The larger the drop, the more likely it is that fluids are accumulating in the device pocket. Hence, the threshold is exceeded if the drop is relatively large. The threshold is not exceeded if the drop is relatively small or if there is no drop. If SVC coil—can Z has dropped significantly, then the detection of possible fluid overload made based on RV TTZ (i.e. RV coil—can Z) at step 306 is deemed to be a false positive since the fluid index crossing may have been due to the increase in pocket fluids rather than any increase in pulmonary fluids. Conversely, if SVC coil—can Z has not dropped significantly, then the detection of the fluid overload is deemed to be reliable (at least insofar as pocket fluids are concerned.) Accordingly, following step 320, if no significant drop in SVC coil—can Z has been detected, then the indication of a possible pulmonary fluid overload initially made at step 306 is deemed to be confirmed or corroborated at step 322 (or at least has not been disconfirmed.) Thereafter, at step 324, the device responds by generating a pulmonary edema/fluid overload warning, etc. On the other hand, if a significant drop in SVC coil—can Z was detected, then the indication of a possible pulmonary fluid overload made at step 306 is deemed to be disconfirmed at step 326 (or at least not corroborated.) At step 328, the device may generate a warning of possible pocket fluid accumulation in view of the drop in SVC coil—can Z.

Note that value for the SVC coil—can drop threshold can be initially set, for example, to a default value set such as −10%. The threshold can thereafter be adjusted based on SVC coil—can values collected within the patient prior to known and verified pulmonary edema/fluid overloads. For patients with device replacement, the baseline from pre-surgery can be measured to access fluid in pocket due to surgery. Note also that, rather than employ SVC coil—can Z as a proxy for pocket fluids, the device might instead measure or estimate pocket fluids using other techniques, if so equipped. Also, techniques described in U.S. patent application Ser. No. 13/007,424 of Gutfinger et al., filed Jan. 14, 2011, entitled “Systems and Methods for Exploiting Near-Field Impedance and Admittance for use with Implantable Medical Devices” may be used to assess device pocket fluids via a near-field interpretation of impedance measurements. See, also, U.S. patent application Ser. No. 12/853,130 of Gutfinger et al., filed Aug. 9, 2010, also entitled “Near Field-Based Systems and Methods for Assessing Impedance and Admittance for use with an Implantable Medical Device” and U.S. patent application Ser. No. 13/007,424, filed Jan. 14, 2011, which is a CIP thereof. The near-field techniques discussed in these patent applications may be generally applicable to any of the impedance-based measurement and interpretation techniques described herein.

One particular example of the pocket fluid-based corroboration technique of FIG. 4 is summarized as follows:

Store baseline SVC coil to Can Z following device implant:
Measure SVC coil to can Z timed at peak of QRS for several beats over respiration cycles.
Store one-day averaged Z of SVC coil—can

Monitoring SVC coil—can Z post-implant:
At the time of taking RV TTZ, measure SVC coil—can Z
Store daily average SVC coil—can Z over several days that can be plotted on a programmer screen for use a surrogate of pocket fluid
If RV TTZ is crossing threshold post-implant (i.e. post-surgery), check the SVC coil—can Z changes. Post-surgery, pocket fluid should gradually diminish as this component from SVC coil—can Z should increase. If SVC coil—can Z curve over several days goes up and then comes down, pocket fluid accumulation is indicated. If a drop in SVC coil—can Z post-surgery is large compared to the SVC coil—can Z baseline and RV IACZ at the time of crossing right after implant, a warning would be false positive.

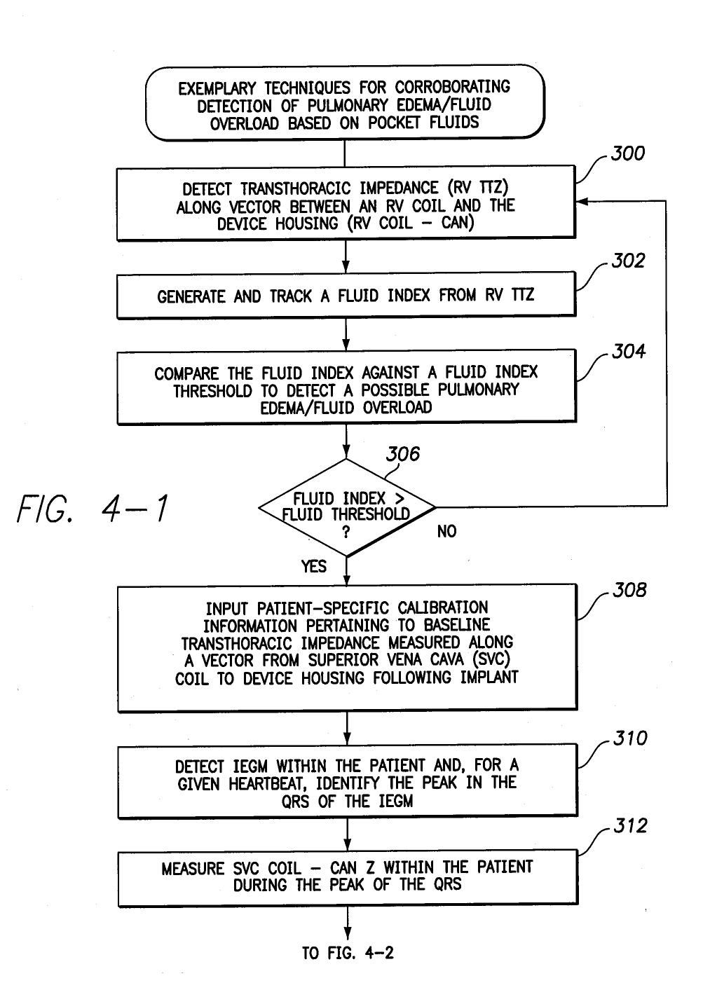

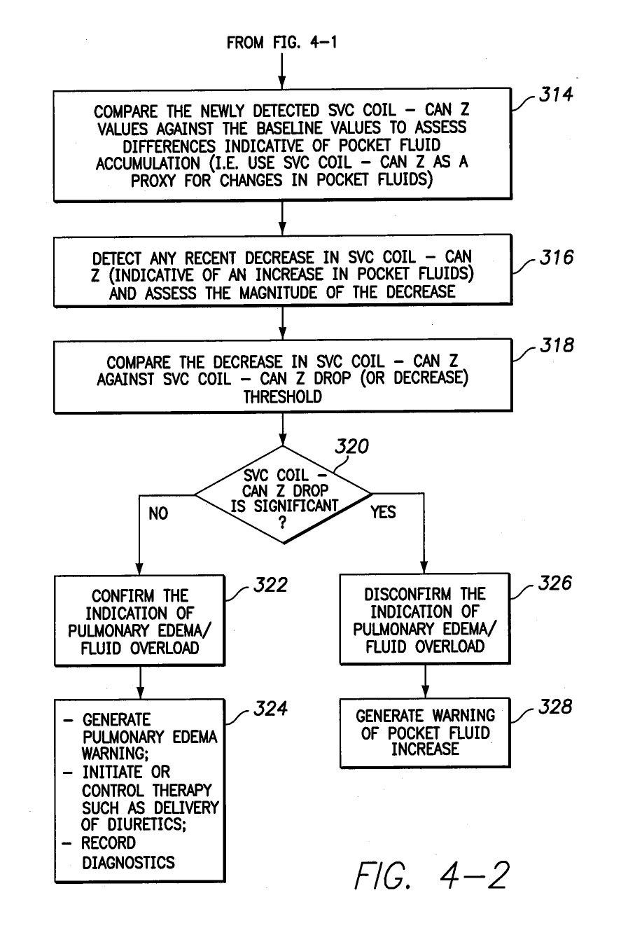

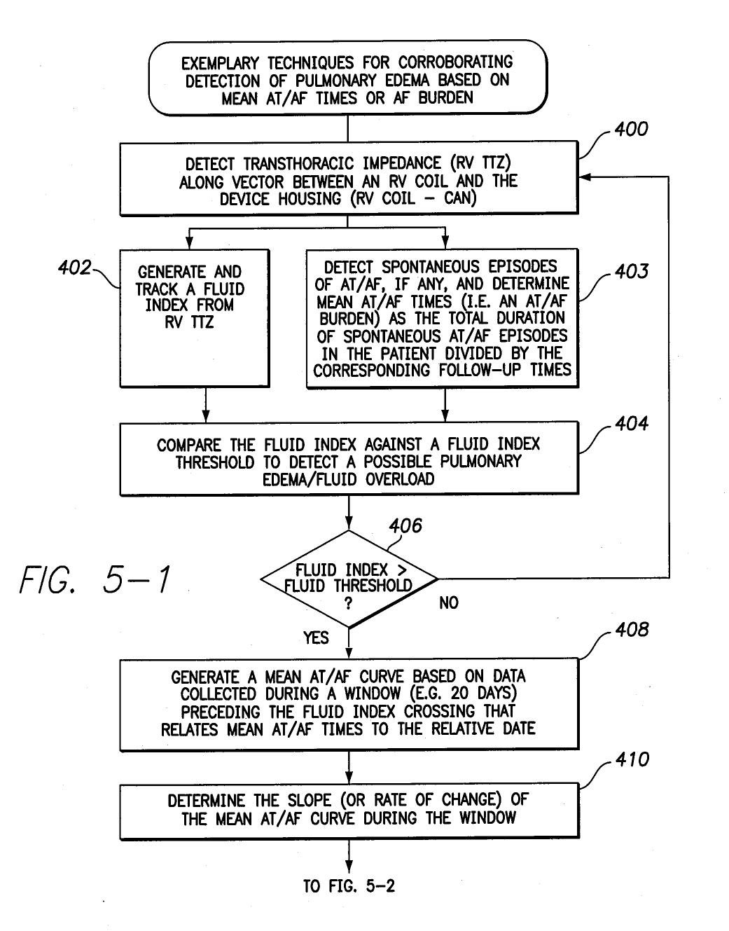

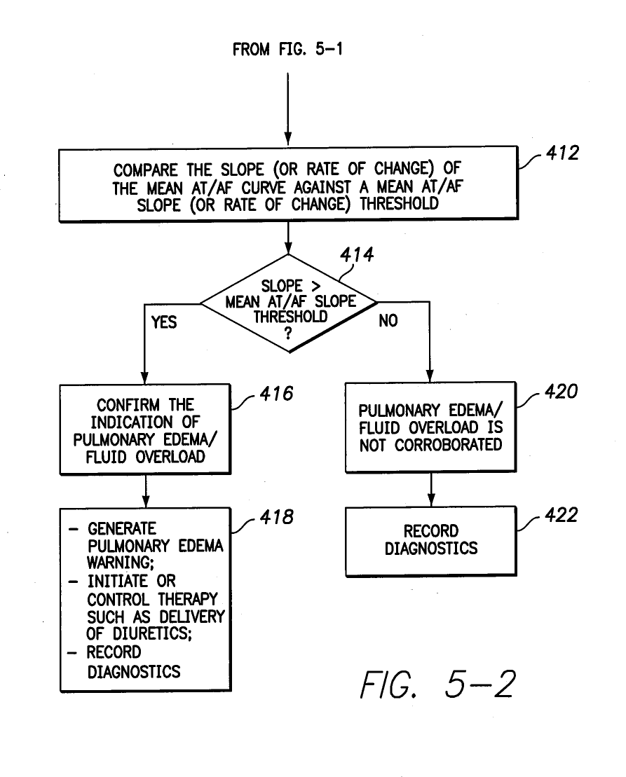

FIG. 5 illustrates corroboration techniques that exploit mean AT/AF times. Some aspects of the technique are the same or similar to those already described and hence will not be described again in detail. At step 400, the device detects TTZ along the RV coil—can vector and, at step 402, generates and tracks the fluid index. Concurrently, at step 403, the device detects episodes of AT/AF, if any. For each episode, the device measures and records the time or duration of the episode. AT/AF may be detected using otherwise conventional techniques such as by detecting an atrial rate and comparing the rate against an AT/AF rate threshold. The duration of individual episodes of AT/AF may be determined based on the start and stop times of the episodes (as detected using crossings of the AT/AF rate threshold.) As noted, the mean AT/AF time may be calculated as the total duration of spontaneous AT/AF episodes in the patient divided by the corresponding follow-up time. This may also be regarded as an “AT/AF burden.”

At step 404, the device compares the fluid index against the fluid index threshold to detect a possible pulmonary edema/fluid overload. Assuming that the fluid index has risen above the threshold (as determined at step 406) then the device at step 408 examines the AT/AF data collected at step 403. In one example, the device examines AT/AF data collected during the twenty days preceding the fluid index crossing detected at step 406. In other examples, a window of different duration can be used, such as a sixty day window. Based on the AT/AF data within the selected window, the device generates an AT/AF curve that relates mean AT/AF times to the relative date within the window. That is, for each day within the designated time window, the device calculates an average or mean AT/AF time based on the AT/AF data collected during that particular day. The device then generates an AT/AF curve based on that data.

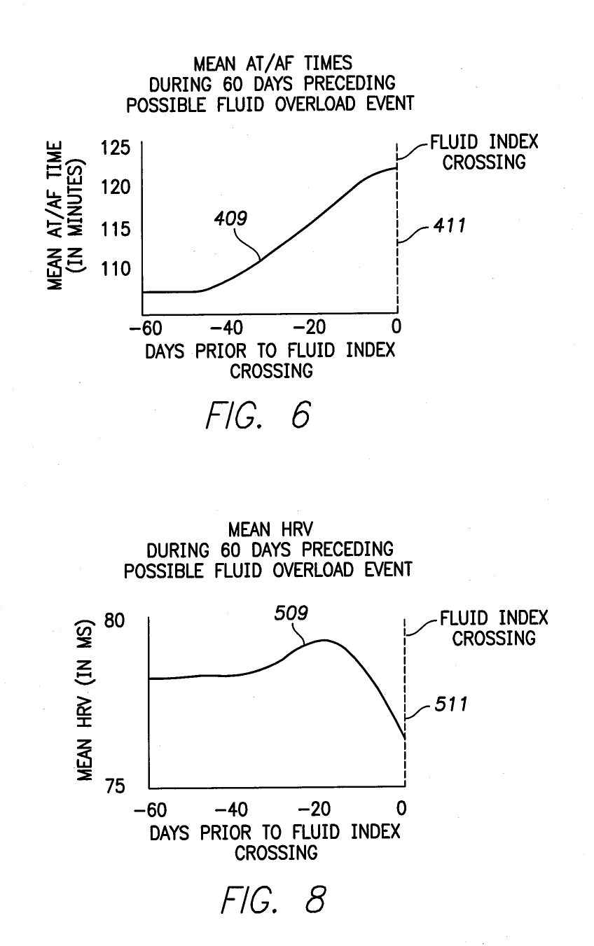

FIG. 6 illustrates an exemplary AT/AF curve 409 for a sixty day window preceding a fluid index crossing 411. This is a stylized representation showing a smooth curve. Actual data may be noisier. (See, for example, data presented in an Abstract by Andriulli et al., “Temporal Association between Fluid Accumulation and Atrial and Ventricular Tachyarrhythmias: An Analysis of 46,696 CRT-D and ICD Patients”, Sep. 19, 2011, HFSA 2011, cited as HFSA 2011-9-19 #162.) As can be seen within FIG. 6, the mean AT/AF time is relatively low at the beginning of the window (under one hundred ten minutes) but increases in duration as the fluid index crossing event approaches (until it is over one hundred twenty minutes.) As such, the upward slope of the curve (especially within the twenty days preceding the fluid index crossing) is relatively significant.

Returning to FIG. 4, at step 410 the device calculates or determines the slope (or rate of change) of the mean AT/AF curve during the selected window, such as by performing linear regression on the collected mean AT/AF time data. At step 412, the device then compares the slope in the mean AT/AF curve against a mean AT/AF slope threshold. If the slope exceeds a threshold indicative of a significant positive slope (i.e. the mean AT/AF time was increasing prior to the possible fluid overload detected at step 406), then the detection of the fluid overload is corroborated since mean AT/AF times were increasing prior to the event in a manner consistent with an expectations. Conversely, if the slope is not above the threshold (i.e. the mean AT/AF curve was not increasing fast enough or not at all), then the detection of the fluid overload is deemed to be unreliable since mean AT/AF times did not increase as expected prior to the event. Accordingly, following step 414, if the mean AT/AF slope threshold was exceeded, then the indication of a possible pulmonary fluid overload is deemed to be confirmed or corroborated at step 416 (or at least has not been disconfirmed.) Thereafter, at step 418, the device responds by generating a pulmonary edema/fluid overload warning, etc. On the other hand, if the mean AT/AF slope threshold was not exceeded, then the indication of a possible pulmonary fluid overload made at step 406 is not corroborated. At step 428, the device may record diagnostics indicating that a fluid overload was tentatively detected but not corroborated based on mean AT/AF times.

Note that value for the mean AT/AF curve slope threshold can be initially set by, for example, to a slope greater than 0.3 within patient for consecutive three days in units of minutes and days. The threshold can thereafter be adjusted based on mean AT/AF data collected within the patient prior to known and verified pulmonary edema/fluid overloads.

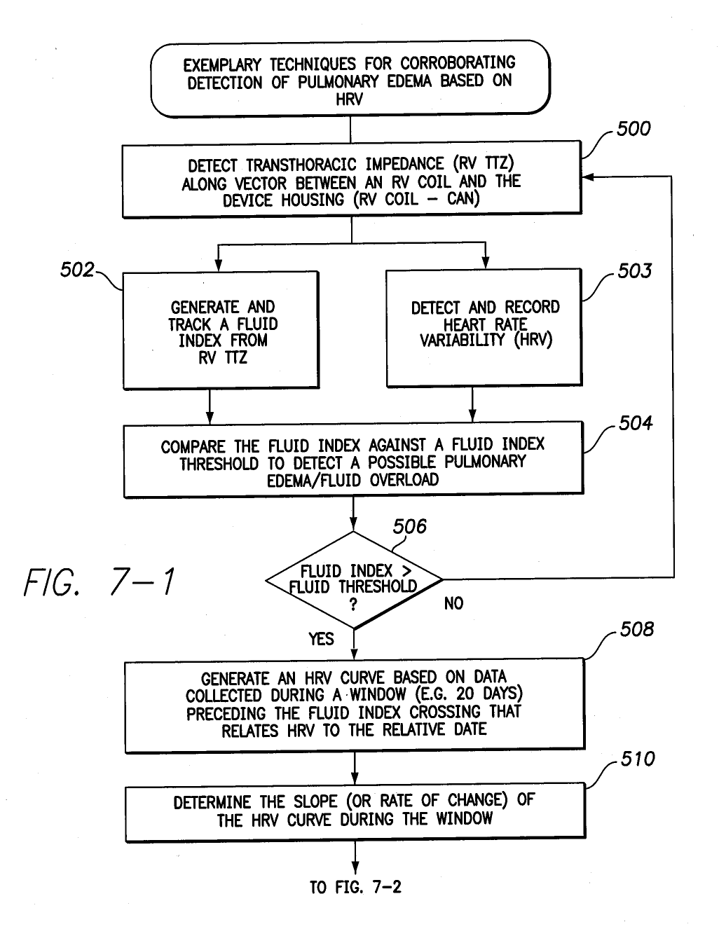

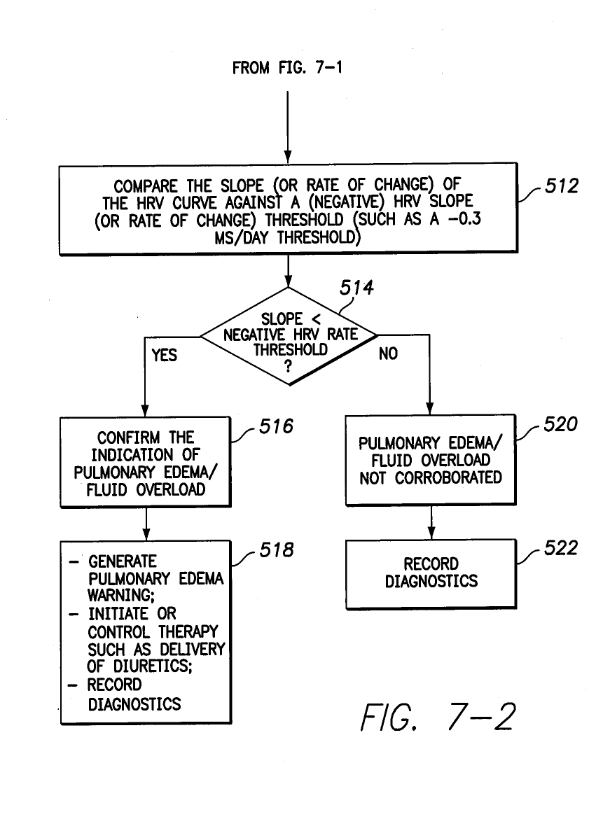

FIG. 7 illustrates corroboration techniques that exploit HRV. Some aspects of the technique are the same or similar to those already described and hence will not be described again in detail. At step 500, the device detects RV TTZ along the RV coil—can vector and, at step 502, generates and tracks the fluid index. Concurrently, at step 503, the device detects HRV. HRV is discussed in U.S. Pat. No. 6,480,733 to Turcott, entitled “Method for Monitoring Heart Failure.” At step 504, the device compares the fluid index against the fluid index threshold to detect a possible pulmonary edema/fluid overload. Assuming that the fluid index has risen above the threshold (as determined at step 506) then the device at step 508 examines the HRV data collected at step 503, such as data collected during the twenty days preceding the fluid index crossing. In other examples, a window of different duration might be used. Based on the HRV data within the selected window, the device generates an HRV curve that relates HRV to the relative date within the window. That is, for each day within the designated time window, the device calculates an average or mean HRV based on the data collected during that particular day. The device then generates an HRV curve based on that data.

FIG. 8 illustrates an exemplary HRV curve 509 for a sixty day window preceding a fluid index crossing 511. This is a stylized representation showing a smooth curve. Actual data may be noisier. (See, again, data presented by Andriulli et al., cited above.) As can be seen, the HRV drops significantly as the fluid overload approaches. As such, the downward slope of the curve (especially within the twenty days preceding the fluid index crossing) is relatively significant.

Returning to FIG. 7, at step 510 the device calculates or determines the slope (or rate of change) of the HRV curve during the selected window, such as by performing linear regression on the collected mean HRV data. At step 512, the device then compares the slope in the HRV curve against a negative slope threshold (i.e. a threshold representative of a significant downward trend in HRV). If the slope is less than the negative threshold (indicative of a significant negative slope in HRV), then the detection of the fluid overload is corroborated since HRV was decreasing prior to the event in a manner consistent with an expectations. Conversely, if the slope is above the threshold (i.e. the HRV curve was not decreasing fast enough or not at all), then the detection of the fluid overload is deemed to be unreliable since mean HRV times did not decrease as expected prior to the event. Accordingly, following step 514, if the HRV slope was sufficiently negative, then the indication of a possible pulmonary fluid overload is deemed to be confirmed or corroborated at step 516 (or at least has not been disconfirmed.) Thereafter, at step 518, the device responds by generating a pulmonary edema/fluid overload warning, etc. On the other hand, if the HRV slope was not sufficiently negative, then the indication of a possible pulmonary fluid overload made at step 506 is not corroborated. At step 528, the device may record diagnostics indicating that a fluid overload was tentatively detected but not corroborated based on HRV.

Note that value for the HRV curve slope threshold can be initially set by, for example, to a default value set such as −50%. Alternatively, the threshold can be set to a slope less than −0.3 where the units are in milliseconds (ms) per day sustained over three days. The threshold can thereafter be adjusted based on HRV data collected within the patient prior to known and verified pulmonary edema/fluid overloads.

One example of the AT/AF and HRV corroboration techniques of FIGS. 5-8 is summarized as follows:
At the time of fluid index threshold crossing, exam the mean time in AT/AF and HRV curves back 10-20 days
If the slope of the mean AT/AF curve>threshold_A (>0) for consecutive five days or more, true positive is confirmed.
If the slope of the mean HRV curve<threshold_H (<0) for consecutive five days or more, true positive is confirmed.
   The techniques using mean AT/AF and HRV can also be used to manage patients earlier for reducing heart failure hospitalizations. For example, if mean AT/AF time increases or HRV decreases (slope of change>threshold) for five days, the patient can be notified to seek treatment in a doctor's office so as to reduce the chance of subsequent hospitalization due to a fluid overload.

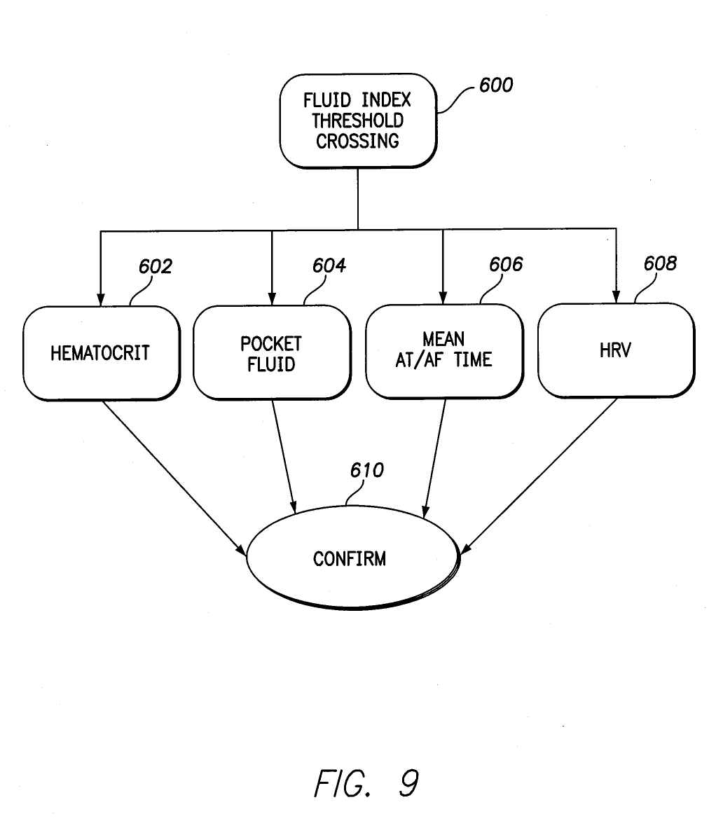

FIG. 9 shows that the various corroboration techniques so far described can be combined. Briefly, at step 600, a fluid index crossing is detected using techniques already described. At steps 600, 602, 604, 606 and 608 the corroborative parameters discussed above are evaluated (i.e. hematocrit (via impedance proxy), pocket fluid (via impedance proxy), mean AT/AF time trends and HRV trends) and combined to confirm 610 or disconfirm the pulmonary edema/fluid overload. For example, a single metric value may be generated representative of a combination of the corroborative parameters for comparison against a suitable metric threshold. In some examples, the individual corroborative parameters may be weighted separately to, for example, emphasize some of the corroborative parameters more so than others. Techniques for generating a combined metric based on various parameters for evaluation are discussed in: U.S. Pat. No. 7,207,947 to Koh et al., entitled “System and Method for Detecting Circadian States Using an Implantable Medical Device.”

In the following, various techniques for detecting changes in hematocrit or pocket fluids are described that may be used alone or in combination with the corroborative techniques discussed above.

### Hematocrit and Pocket Fluid Assessment Techniques

FIG. 10 broadly summarizes a technique for assessing hematocrit based on intracardiac impedance. Hematocrit assessment techniques were already described in detail with reference to the corroborative technique of FIG. 3 above and hence will only be briefly summarized here to illustrate that the techniques can be used as a standalone assessment of hematocrit. At step 700, the implantable device inputs patient-specific calibration information relating baseline hematocrit to baseline RV IACZ measured, e.g., RV coil—RV ring or RV tip—RV ring at the peak of the QRS of the IEGM. As explained above, this data can be collected post-implant, then stored within the device. At step 702, the device measures RV IACZ within the patient, also at the peak of the QRS of the IEGM. At step 704, the device generates a proxy for the hematocrit of the patient and/or detects a significant change in hematocrit by comparing the measured RV IACZ against the patient-specific calibration information relating hematocrit to RV IACZ for the patient. At step 706, if the proxy for hematocrit (or a detected change in hematocrit) is outside acceptable bounds, the device generates a suitable warning, initiates or controls therapy, records diagnostics or controls other device functions.

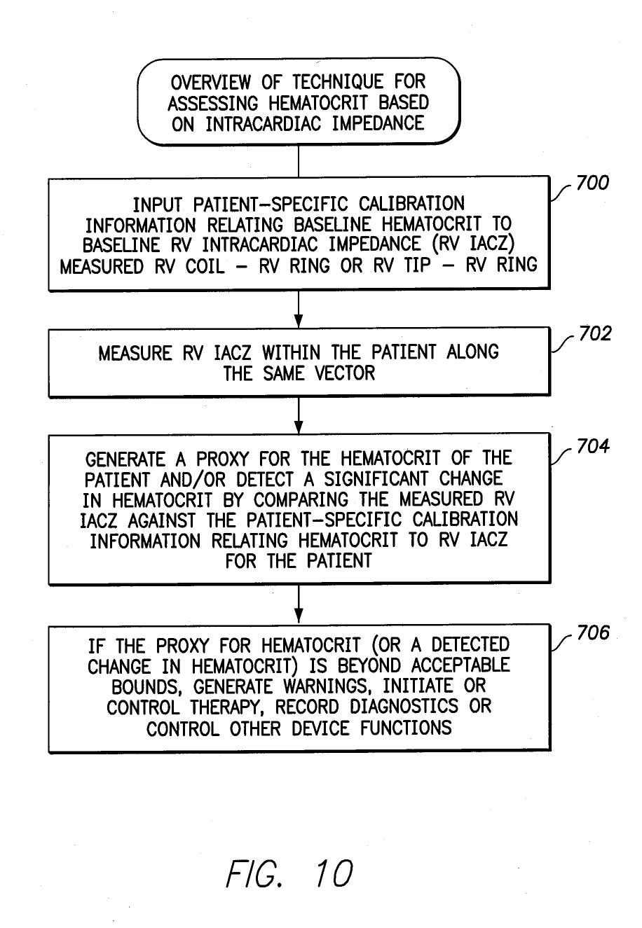

FIG. 11 broadly summarizes a technique for assessing pocket fluids based on SVC coil—can impedance. Pocket fluid assessment techniques were already described in detail with reference to the corroborative technique of FIG. 4 above and hence will only be briefly summarized here to illustrate that the techniques can be used as a standalone assessment of pocket fluids. At step 800, the implantable device inputs patient-specific calibration information pertaining to baseline transthoracic impedance measured SVC coil—can impedance at the peak of the QRS of the IEGM. As explained, this data can be collected post-implant, then stored within the device. At step 802, the device measures SVC coil—can Z within the patient, also at the peak of the QRS of the IEGM. At step 804, the device generates a proxy for pocket fluid accumulation and/or detects a significant change in accumulation by comparing the measured SVC coil—can Z against the patient-specific calibration information. At step 806, if the accumulation proxy (or a change in accumulation) is outside acceptable bounds, the device generates a suitable warning, initiates or controls therapy, records diagnostics or controls other device functions.

In the following, various techniques for predicting a pulmonary edema/fluid overload event are described.

### AT/AF and HRV-Based Predictive Assessment Techniques

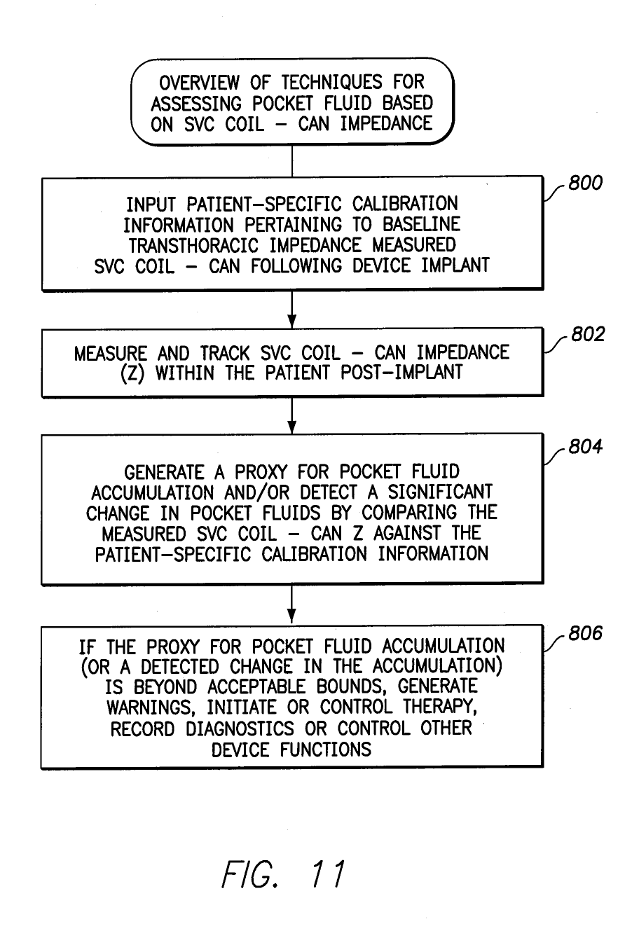

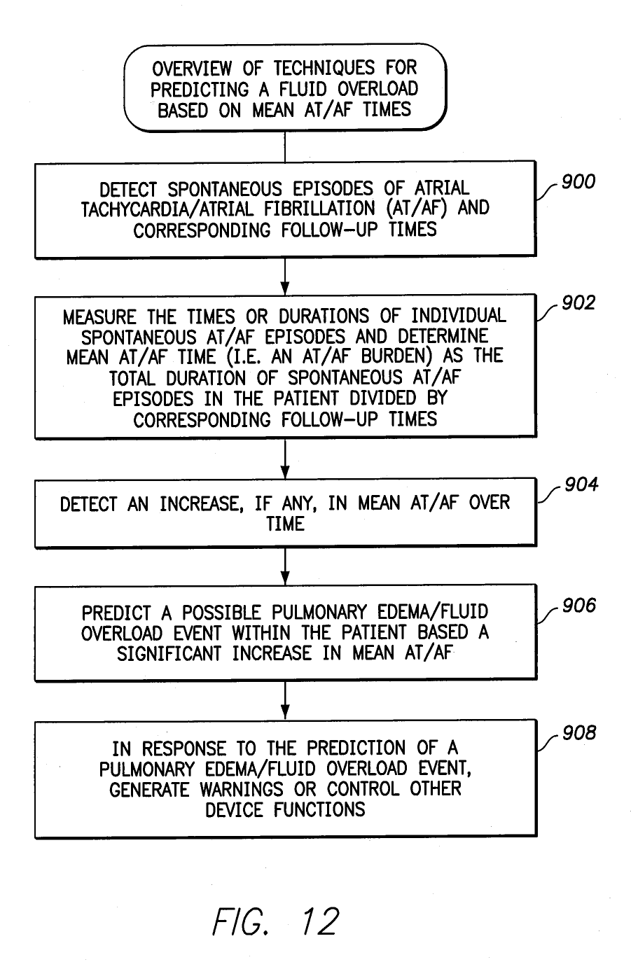

FIG. 12 broadly summarizes a technique for predicting a possible pulmonary edema/fluid overload event based on mean AT/AF times. Related techniques were described in detail above with reference to the corroborative methods of FIGS. 5 and 6. However, the techniques of FIG. 12 are intended to be predictive not corroborative. At step 900, the implantable device detects spontaneous episodes of AT/AF and corresponding follow-up times. At step 902, the device measures the times or durations of individual spontaneous AT/AF episodes and determines mean AT/AF times (i.e. AT/AF burden) as the total duration of spontaneous AT/AF episodes in the patient divided by the corresponding follow-up times. At step 904, the device detects an increase, if any, in means AT/AF over time. If mean AT/AF is increasing, this may be an indication of an imminent pulmonary edema/fluid overload event. Accordingly, at step 906, the device predicts a possible pulmonary edema/fluid overload event within the patient based a significant increase in mean AT/AF (as detected using a suitable threshold.) By predicting a possible pulmonary edema/fluid overload event, it is meant that device generates an indication of an increased likelihood of such an event. As can be appreciated, such predictions are not absolute but are probabilistic. Nevertheless, an indication of an increased likelihood of an event can be useful for warning the patient or caregiver, or for initiating prophylactic measures. Accordingly, in response to the prediction, the device at step 908 generates warnings or controls other device functions.

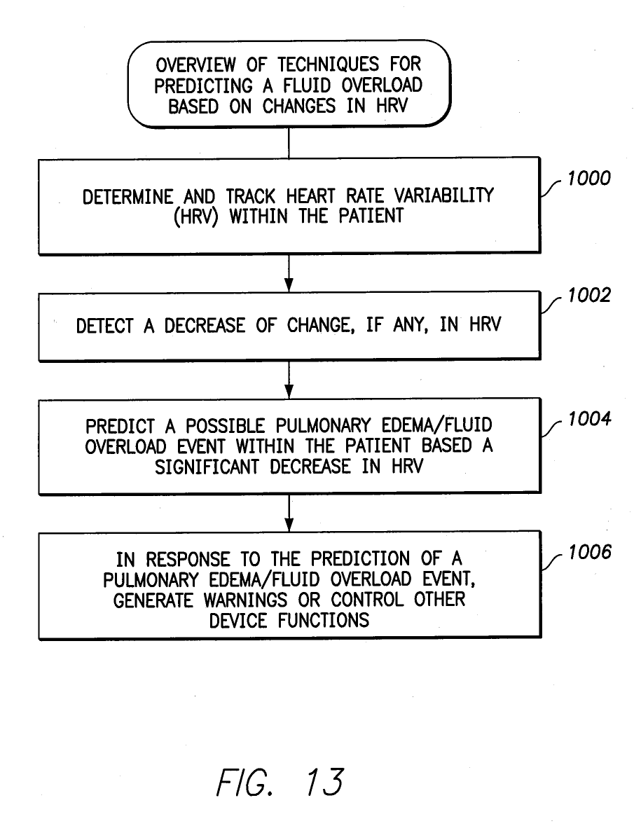

FIG. 13 broadly summarizes a technique for predicting a possible pulmonary edema/fluid overload event based on HRV. Related techniques were described in detail above with reference to the corroborative methods of FIGS. 7 and 8. The techniques of FIG. 13 are intended to be predictive not corroborative. At step 1000, the implantable device determines and tracks HRV within the patient and, at step 1002, detects a decrease, if any, in the HRV over time. This may be done by averaging HRV each day and then recording the averaged or mean HRV each day for comparison with preceding values. If HRV is decreasing, this may be an indication of an imminent pulmonary edema/fluid overload event. Accordingly, at step 1006, the device predicts a possible pulmonary edema/fluid overload event within the patient based a significant decrease in HRV (as detected using a suitable HRV threshold.) As explained above, by prediction it is meant that device generates an indication of an increased likelihood of such an event. In response to the prediction, the device at step 1006 generates warnings or controls other device functions.

Thus various systems and methods have been described including, but not limited to: a) systems and methods for determining hematocrit changes through intra-cardiac Z; b) systems and methods for determining device pocket fluid through SVC-CAN Z; c) systems and methods of using mean AT/AF time and HRV as precursor for fluid index threshold crossing; and d) systems and methods for reducing false positive events with fluid index threshold crossing by confirming with hematocrit, pocket fluid, mean AF/AT time and HRV.

For the sake of completeness, a detailed description of an exemplary pacer/CRT for performing the techniques described above will now be provided. However, principles of invention may be implemented within other pacer/CRT implementations or within other implantable devices such as stand-alone monitoring devices or ICDs. Furthermore, although examples described herein involve processing of impedance and other data by the implanted device itself, some operations may be performed using an external device, such as a bedside monitor, device programmer, computer server or other external system. For example, impedance parameters might be transmitted to the external device, which processes the data to corroborate detection of a pulmonary fluid overload. Processing by the implanted device itself is preferred as that allows the device to detect events more promptly and to take appropriate action.

Note also that the techniques described herein may be selectively combined with, or corroborated by, other pulmonary fluid monitoring techniques, where appropriate. See, for example, U.S. Pat. No. 7,272,443 of Min et al., entitled “System and Method for Predicting a Heart Condition based on Impedance Values using an Implantable Medical Device”; U.S. Pat. No. 8,032,212 of Bornzin et al., entitled “System and Method for Monitoring Thoracic Fluid Levels Based on Impedance Using an Implantable Medical Device”; and U.S. Pat. No. 7,917,194 to Reed et al., entitled “Method and Apparatus for Detecting Pulmonary Edema.”

### Exemplary Pacer/ICD

FIG. 13 provides a simplified block diagram of the pacer/ICD, which is a dual-chamber stimulation device capable of treating both fast and slow arrhythmias with stimulation therapy, including cardioversion, defibrillation, and pacing stimulation, as well as capable of performing the pulmonary fluid monitoring functions described above. To provide atrial chamber pacing stimulation and sensing, pacer/ICD 10 is shown in electrical communication with a heart 1112 by way of a left atrial lead 1120 having an atrial tip electrode 1122 and an atrial ring electrode 1123 implanted in the atrial appendage. Pacer/ICD 10 is also in electrical communication with the heart by way of a right ventricular lead 1130 having, in this embodiment, a ventricular tip electrode 1132, a right ventricular ring electrode 1134, a right ventricular (RV) coil electrode 1136, and a superior vena cava (SVC) coil electrode 1138. Typically, the right ventricular lead 1130 is transvenously inserted into the heart so as to place the RV coil electrode 1136 in the right ventricular apex, and the SVC coil electrode 1138 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a “coronary sinus” lead 1124 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 1124 is designed to receive atrial and ventricular cardiac signals and to deliver left ventricular pacing therapy using at least a left ventricular tip electrode 1126, left atrial pacing therapy using at least a left atrial ring electrode 1127, and shocking therapy using at least a left atrial coil electrode 1128. With this configuration, biventricular pacing can be performed. Although only three leads are shown in FIG. 13, it should also be understood that additional stimulation leads (with one or more pacing, sensing and/or shocking electrodes) might be used in order to efficiently and effectively provide pacing stimulation to the left side of the heart or atrial cardioversion and/or defibrillation.

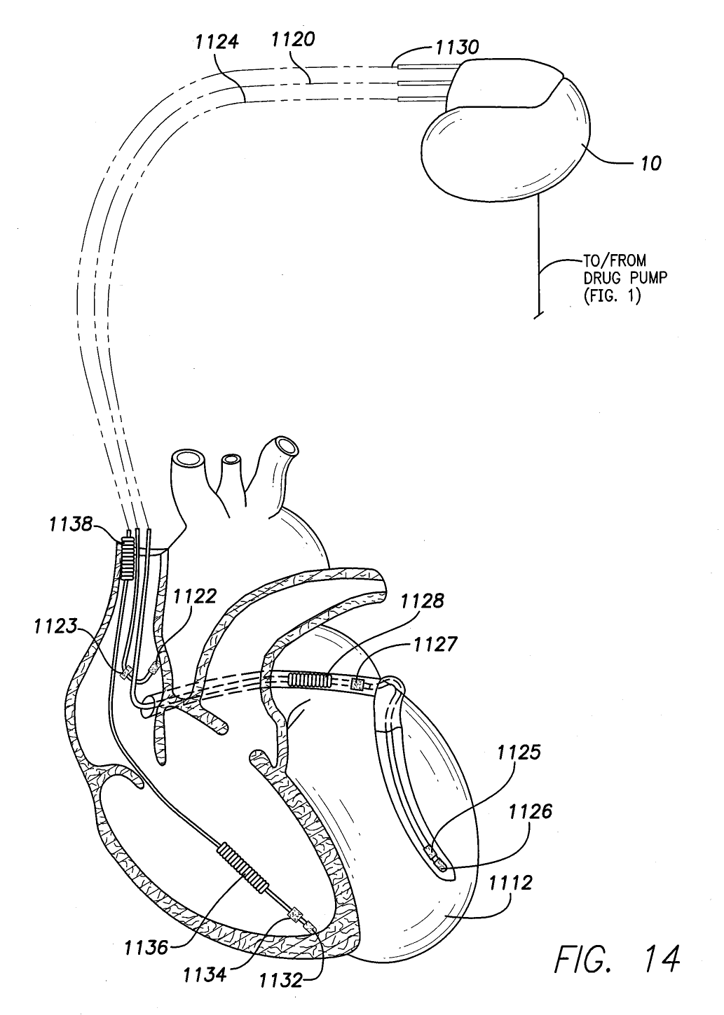

A simplified block diagram of internal components of pacer/ICD 10 is shown in FIG. 14. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned impedance-based functions.

The housing 1140 for pacer/ICD 10, shown schematically in FIG. 14, is often referred to as the “can”, “case” or “case electrode” and may be programmably selected to act as the return electrode for all “unipolar” modes. The housing 1140 may further be used as a return electrode alone or in combination with one or more of the coil electrodes, 1128, 1136 and 1138, for shocking purposes. The housing 1140 further includes a connector (not shown) having a plurality of terminals, 1142, 1143, 1144, 1146, 1148, 1152, 1154, 1156 and 1158 (shown schematically and, for convenience, the names of the electrodes to which they are connected are shown next to the terminals). As such, to achieve right atrial sensing and pacing, the connector includes at least a right atrial tip terminal (A_{R} TIP) 1142 adapted for connection to the atrial tip electrode 1122 and a right atrial ring (A_{R} RING) electrode 1143 adapted for connection to right atrial ring electrode 1123. To achieve left chamber sensing, pacing and shocking, the connector includes at least a left ventricular tip terminal (V_{L} TIP) 1144, a left atrial ring terminal (A_{L} RING) 1146, and a left atrial shocking terminal (A_{L} COIL) 1148, which are adapted for connection to the left ventricular ring electrode 1126, the left atrial tip electrode 1127, and the left atrial coil electrode 1128, respectively. To support right chamber sensing, pacing and shocking, the connector further includes a right ventricular tip terminal (V_{R} TIP) 1152, a right ventricular ring terminal (V_{R} RING) 1154, a right ventricular shocking terminal (R_{V} COIL) 1156, and an SVC shocking terminal (SVC COIL) 1158, which are adapted for connection to the right ventricular tip electrode 1132, right ventricular ring electrode 1134, the RV coil electrode 1136, and the SVC coil electrode 1138, respectively. Although not shown, additional terminals may be needed for use with drug pump 14.

At the core of pacer/ICD 10 is a programmable microcontroller 1160, which controls the various modes of stimulation therapy. As is well known in the art, the microcontroller 1160 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 1160 includes the ability to process or monitor input signals (data) as controlled by a program code stored in a designated block of memory. The details of the design and operation of the microcontroller 1160 are not critical to the invention. Rather, any suitable microcontroller 1160 may be used that carries out the functions described herein. The use of microprocessor-based control circuits for performing timing and data analysis functions are well known in the art.

As shown in FIG. 14, an atrial pulse generator 1170 and a ventricular/impedance pulse generator 1172 generate pacing stimulation pulses for delivery by the right atrial lead 1120, the right ventricular lead 1130, and/or the coronary sinus lead 1124 via an electrode configuration switch 1174. It is understood that in order to provide stimulation therapy in each of the four chambers of the heart, the atrial and ventricular pulse generators, 1170 and 1172, may include dedicated, independent pulse generators, multiplexed pulse generators or shared pulse generators. The pulse generators, 1170 and 1172, are controlled by the microcontroller 1160 via appropriate control signals, 1176 and 1178, respectively, to trigger or inhibit the stimulation pulses.

The microcontroller 1160 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 1174 includes a plurality of switches for connecting the desired electrodes to the appropriate I/O circuits, thereby providing complete electrode programmability. Accordingly, the switch 1174, in response to a control signal 1180 from the microcontroller 1160, determines the polarity of the stimulation pulses (e.g., unipolar, bipolar, combipolar, etc.) by selectively closing the appropriate combination of switches (not shown) as is known in the art.

Atrial sensing circuits 1182 and ventricular sensing circuits 1184 may also be selectively coupled to the right atrial lead 1120, coronary sinus lead 1124, and the right ventricular lead 1130, through the switch 1174 for detecting the presence of cardiac activity in each of the four chambers of the heart. Accordingly, the atrial (ATR. SENSE) and ventricular (VTR. SENSE) sensing circuits, 1182 and 1184, may include dedicated sense amplifiers, multiplexed amplifiers or shared amplifiers. The switch 1174 determines the “sensing polarity” of the cardiac signal by selectively closing the appropriate switches, as is also known in the art. In this way, the clinician may program the sensing polarity independent of the stimulation polarity. Each sensing circuit, 1182 and 1184, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 1182 and 1184, are connected to the microcontroller 1160 which, in turn, are able to trigger or inhibit the atrial and ventricular pulse generators, 1170 and 1172, respectively, in a demand fashion in response to the absence or presence of cardiac activity in the appropriate chambers of the heart.

For arrhythmia detection, pacer/ICD 10 utilizes the atrial and ventricular sensing circuits, 1182 and 1184, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 1160 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 1190. The data acquisition system 1190 is configured to acquire intracardiac electrogram signals, convert the raw analog data into a digital signal, and store the digital signals for later processing and/or telemetric transmission to an external device 1202. The data acquisition system 1190 is coupled to the right atrial lead 1120, the coronary sinus lead 1124, and the right ventricular lead 1130 through the switch 1174 to sample cardiac signals across any pair of desired electrodes. The microcontroller 1160 is further coupled to a memory 1194 by a suitable data/address bus 1196, wherein the programmable operating parameters used by the microcontroller 1160 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 1194 through a telemetry circuit 1200 in telemetric communication with the external device 1202, such as a programmer, transtelephonic transceiver or a diagnostic system analyzer. The telemetry circuit 1200 is activated by the microcontroller by a control signal 1206. The telemetry circuit 1200 advantageously allows intracardiac electrograms and status information relating to the operation of pacer/ICD 10 (as contained in the microcontroller 1160 or memory 1194) to be sent to the external device 1202 through an established communication link 1204. Pacer/ICD 10 further includes an accelerometer or other physiologic sensor 1208, commonly referred to as a “rate-responsive” sensor because it is typically used to adjust pacing stimulation rate according to the exercise state of the patient. However, the physiological sensor 1208 may further be used to detect changes in cardiac output, changes in the physiological condition of the heart, or diurnal changes in activity (e.g., detecting sleep and wake states) and to detect arousal from sleep. Additionally, sensor 1208 could be equipped to detect pulmonary fluid levels or proxies for pulmonary fluid levels. Accordingly, the microcontroller 1160 responds by adjusting the various pacing parameters (such as rate, AV Delay, V-V Delay, etc.) at which the atrial and ventricular pulse generators, 1170 and 1172, generate stimulation pulses. While shown as being included within pacer/ICD 10, it is to be understood that the physiologic sensor 1208 may also be external to pacer/ICD 10, yet still be implanted within or carried by the patient. A common type of rate responsive sensor is an activity sensor incorporating an accelerometer or a piezoelectric crystal, which is mounted within the housing 1140 of pacer/ICD 10. Other types of physiologic sensors are also known, for example, sensors that sense the oxygen content of blood, respiration rate and/or minute ventilation, pH of blood, ventricular gradient, pulmonary artery pressure, etc.

The pacer/ICD additionally includes a battery 1210, which provides operating power to all of the circuits shown in FIG. 14. The battery 1210 may vary depending on the capabilities of pacer/ICD 10. If the system only provides low voltage therapy, a lithium iodine or lithium copper fluoride cell may be utilized. For pacer/ICD 10, which employs shocking therapy, the battery 1210 must be capable of operating at low current drains for long periods, and then be capable of providing high-current pulses (for capacitor charging) when the patient requires a shock pulse. The battery 1210 must also have a predictable discharge characteristic so that elective replacement time can be detected. Accordingly, pacer/ICD 10 is preferably capable of high voltage therapy and appropriate batteries.

As further shown in FIG. 14, pacer/ICD 10 is shown as having an impedance measuring circuit 1212, which is enabled by the microcontroller 1160 via a control signal 1214. Herein, impedance is primarily detected for use in evaluating thoracic and cardiogenic impedance for use in evaluating thoracic fluids. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; detecting the opening of heart valves; as well as detecting the various impedance parameters described above for corroborating fluid overloads, etc. The impedance measuring circuit 1212 is advantageously coupled to the switch 1174 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 1160 further controls a shocking circuit 1216 by way of a control signal 1218. The shocking circuit 1216 generates shocking pulses of low (up to 0.5 joules), moderate (0.5-10 joules) or high energy (11 to 40 joules or more), as controlled by the microcontroller 1160. Such shocking pulses are applied to the heart of the patient through at least two shocking electrodes, and as shown in this embodiment, selected from the left atrial coil electrode 1128, the RV coil electrode 1136, and/or the SVC coil electrode 1138. The housing 1140 may act as an active electrode in combination with the RV electrode 1136, or as part of a split electrical vector using the SVC coil electrode 1138 or the left atrial coil electrode 1128 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 11-40 joules or more), delivered asynchronously (since R-waves may be too disorganized), and pertaining exclusively to the treatment of fibrillation. Accordingly, the microcontroller 1160 is capable of controlling the synchronous or asynchronous delivery of the shocking pulses.

Microcontroller 1160 also includes various components directed to implementing the aforementioned pulmonary fluid monitoring methods. More specifically, an preliminary RV TTZ-based pulmonary edema/fluid overload detection system 1201 is provided that is operative to detect a possible fluid overload based on the RV coil—can transthoracic impedance detected by impedance measuring circuit 1212. A corroboration parameter determination system 1203 is operative to determine corroborative parameters. Included therein are sub-systems for determining a hematocrit proxy 1205, a device pocket fluid accumulation proxy 1207, a mean AT/AF trend 1209 and an HRV trend 1211. A pulmonary edema/fluid overload confirmation system 1213 is operative to confirm the indication of a fluid overload based on the corroborative parameters and generate an indication of fluid overload in response thereto, using the corroboration techniques described above. Additionally, a pulmonary edema/fluid overload prediction system 1215 is provided that is operative to predict a possible pulmonary edema/fluid overload based on trends in AT/AF durations or HRV using the predictive techniques described above.

A diuresis/therapy/diagnostics controller 1217 controls generation of diagnostic data and warning signals in response to the pulmonary fluid accumulation. Diagnostic data is stored within memory 1194. Warning signals may be relayed to the patient via implanted warning device 1219 or via a bedside monitor, PAM or other external system 16. Controller 1217 also controls and titrates the delivery of diuretics (or other appropriate therapies) using drug pump 14 as described above. In implementations where there is no drug pump, titration of diuretics is typically achieved by instead providing suitable instructions to the patient or caregiver via the bedside monitor (or other external device).

At least some of the techniques described herein may be performed by, or under the control of, the external device. Accordingly, external device 16 is shown to include a pulmonary edema/fluid overload confirmation system 1221 is operative to confirm the detection of a fluid overload based on the corroborative parameters using impedance measurements and other parameters sent from the implanted device. In general, any of the components shown within the microcontroller 1160 may have corresponding components within the external device. Still further, to provide calibration information for use with hematocrit or device pocket fluid accumulations, the external device may include a patient-specific hematocrit and pocket fluid proxy calibration system 1223 to generate or process the patient specific calibration data discussed above.

Depending upon the implementation, the various components of the microcontroller of the implanted device may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

In general, while the invention has been described with reference to particular embodiments, modifications can be made thereto without departing from the scope of the invention. Note also that the term “including” as used herein is intended to be inclusive, i.e. “including but not limited to.”

What is claimed:

1. A method for use with an implantable medical device for implant within a patient, the method comprising:
detecting values representative of transthoracic impedance within the patient;
detecting a possible fluid overload based on the values representative of transthoracic impedance;
detecting corroborative parameters representative of one or more of hematocrit and device pocket fluid accumulation within the patient; and
evaluating the corroborative parameters to confirm the fluid overload and, if confirmed, generating an indication of a fluid overload.

2. The method of claim 1 wherein detecting values representative of transthoracic impedance includes detecting impedance along a vector between a right ventricular (RV) coil electrode and a device housing electrode.

3. The method of claim 1 wherein detecting possible fluid overload includes:
generating a fluid index from the values representative of transthoracic impedance;
comparing the fluid index against a fluid index threshold; and
generating an indication of possible fluid overload if the fluid index rises above the fluid index threshold.

4. The method of claim 1 wherein detecting corroborative parameters is performed to detect parameters representative of hematocrit.

5. The method of claim 4 wherein detecting parameters representative of hematocrit comprises:
inputting patient-specific calibration information relating baseline hematocrit to baseline intracardiac impedance (IACZ) along a vector influenced by hematocrit;
measuring IACZ within the patient along the same vector; and
generating a proxy for the hematocrit of the patient by comparing the measured IACZ against the patient-specific calibration information relating baseline hematocrit to baseline IACZ for the patient.

6. The method of claim 5 wherein the patient-specific calibration information relates baseline hematocrit to baseline IACZ measured at the peak of a ventricular depolarization waveform of an intracardiac electrogram (IEGM) of the patient.

7. The method of claim 6 wherein the measuring IACZ along the same vector within the patient comprises:
detecting IEGM signals within the patient and identifying the peak in a ventricular depolarization waveform of the IEGM; and
measuring IACZ within the patient along the same vector during the peak of the ventricular depolarization waveform of the IEGM.

8. The method of claim 7 wherein generating the proxy for hematocrit comprises:
comparing the IACZ measured during the peak of the ventricular depolarization waveform against the patient-specific calibration information relating baseline hematocrit to baseline IACZ for the patient.

9. The method of claim 5 wherein IACZ is measured using one or more of an RV coil—RV ring vector, an RV tip—RV ring vector and an RA tip—RA ring vector.

10. The method of claim 9 wherein evaluating the corroborative parameters to confirm the fluid overload includes:
detecting any recent drop in measured IACZ;
comparing the drop in measured IACZ against an IACZ drop threshold; and
confirming the fluid overload if the drop in measured IACZ is less than the drop threshold; otherwise disconfirming the fluid overload.

11. The method of claim 10 further including generating a warning signal indicative of anemia if the drop in measured IACZ exceeds the drop threshold.

12. The method of claim 1 wherein detecting corroborative parameters is performed to detect parameters representative of pocket fluid accumulation.

13. The method of claim 12 wherein detecting parameters representative of pocket fluid accumulation comprises:
inputting patient-specific calibration information pertaining to baseline transthoracic impedance along a vector influenced by pocket fluid accumulation;
measuring transthoracic impedance along the same vector;
generating a proxy for pocket fluid accumulation by comparing the measured transthoracic impedance against the patient-specific calibration information.

14. The method of claim 13 wherein the patient-specific calibration information provides baseline transthoracic impedance measured at the peak of a ventricular depolarization waveform of the IEGM of the patient.

15. The method of claim 14 wherein the measuring transthoracic impedance along the same vector within the patient comprises:
detecting IEGM signals within the patient and identifying the peak in a ventricular depolarization waveform of the IEGM; and
measuring transthoracic impedance within the patient along the same vector during the peak of the ventricular depolarization waveform of the IEGM.

16. The method of claim 15 wherein generating the proxy for pocket fluid accumulation comprises:
comparing the transthoracic impedance measured during the peak of the ventricular depolarization waveform against the patient-specific calibration information.

17. The method of claim 13 wherein transthoracic impedance (Z) is measured using a superior vena cava (SVC) coil—can vector.

18. The method of claim 17 wherein evaluating the corroborative parameters to confirm the fluid overload includes:
detecting any recent drop in SVC coil—can Z;
comparing the drop in SVC coil—can Z against an SVC coil—can Z drop threshold; and
confirming the fluid overload if the drop in SVC coil—can Z is less than the drop threshold; otherwise disconfirming the fluid overload.

19. The method of claim 18 further including generating a warning signal indicative of an increase in pocket fluids if the drop in SVC coil—can Z exceeds the drop threshold.

20. The method of claim 1 wherein the corroborative parameters further include parameters representative of atrial tachycardia/atrial fibrillation (AT/AF.)

21. The method of claim 20 wherein detecting parameters representative of AT/AF includes:
detecting spontaneous episodes of AT/AF within the patient;
measuring the durations of the episodes of AT/AF and corresponding follow-up times;
determining a mean AT/AF time based on a total duration of spontaneous AT/AF episodes in the patient divided by the corresponding follow-up times; and
determining a rate of change, if any, in mean AT/AF times during an interval of time preceding the possible fluid overload.

22. The method of claim 21 wherein evaluating the corroborative parameters to confirm the fluid overload includes:
comparing the rate of change in mean AT/AF against a mean AT/AF rate threshold; and
confirming the indication of fluid overload if the rate of change exceeds the threshold.

23. The method of claim 1 wherein the corroborative parameters further include parameters representative of heart rate variability (HRV.)

24. The method of claim 23 wherein detecting parameters representative of HRV includes determining a rate of change, if any, in HRV during an interval of time preceding the possible fluid overload.

25. The method of claim 24 wherein evaluating the corroborative parameters to confirm the fluid overload includes:
comparing the rate of change in HRV against an HRV rate threshold; and
confirming the indication of fluid overload if the rate of change does not exceed the threshold.

26. The method of claim 1 wherein, if the fluid overload is confirmed, controlling delivery of therapy in response thereto.

27. The method of claim 1 wherein, if the fluid overload is confirmed, generating warning signals in response thereto.

28. The method of claim 1 wherein all of the steps are performed by the implantable medical device.

29. The method of claim 1 wherein at least some of the steps are performed by an external device in communication with the implantable medical device.

30. A system for use with an implantable medical device for implant within a patient, the system comprising:
a transthoracic impedance detection system operative to detect transthoracic impedance within the patient;
a preliminary fluid overload detection system operative to detect a possible fluid overload based on the transthoracic impedance;
a corroborative parameter determination system operative to determine corroborative parameters representative of one or more of hematocrit and device pocket fluid accumulation within the patient; and
a fluid overload confirmation system operative to confirm the indication of a fluid overload based on the corroborative parameters and to generate an indication of fluid overload in response thereto.

31. A system for use with an implantable medical device for implant within a patient, the system comprising:
means for detecting transthoracic impedance within the patient;
means for detecting possible fluid overload based on the transthoracic impedance;
means for determining corroborative parameters representative of one or more of hematocrit and device pocket fluid accumulation within the patient; and
means for confirming the indication of fluid overload based on the corroborative parameters.

32. A method for use with an implantable medical device for implant within a patient, the method comprising:
inputting patient-specific calibration information relating baseline hematocrit to baseline right ventricular (RV) intracardiac impedance (IACZ) along an RV vector influenced by hematocrit;
measuring RV IACZ within the patient along the same RV vector;
generating a proxy for the hematocrit of the patient by comparing the measured RV IACZ against the patient-specific calibration information relating baseline hematocrit to baseline RV IACZ for the patient; and
controlling at least one device function based on the proxy for hematocrit.

33. A method for use with an implantable medical device for implant within a patient, the method comprising:
inputting patient-specific calibration information pertaining to baseline transthoracic impedance along a superior vena cava (SVC) coil—can vector;
measuring SVC coil—can transthoracic impedance;
generating a proxy for pocket fluid accumulation by comparing the measured SVC coil—can transthoracic impedance against the patient-specific calibration information; and
controlling at least one device function based on the proxy for pocket fluid accumulation.

34. A method for use with an implantable medical device for implant within a patient, the method comprising:
detecting spontaneous episodes of atrial tachycardia/atrial fibrillation (AT/AF) within the patient;
measuring the durations of the spontaneous episodes of AT/AF and corresponding follow-up times;
determining mean AT/AF times based on a total duration of spontaneous AT/AF episodes in the patient divided by the corresponding follow-up times;
detecting a rate of change, if any, in the mean AT/AF times; and
predicting a fluid overload within the patient based on the rate of change in mean AT/AF times.

35. A method for use with an implantable medical device for implant within a patient, the method comprising:
detecting heart rate variability (HRV) within the patient;
detecting a rate of change, if any, in HRV; and
predicting a fluid overload within the patient based on the rate of change in HRV.
